(19) United States
(12) Patent Application Publication
(10) Pub. No.: US 2016/0052937 A1
(43) Pub. Date: Feb. 25, 2016

(54) Tetrahydroimidazo[1,5-d][1,4]oxazepine compound

(71) Applicant: Eisai R&D Management Co., Ltd., Tokyo (JP)

(72) Inventors: Mamoru Takaishi, Tsukuba (JP); Nobuhiro Sato, Tsukuba (JP); Takafumi Motoki, Tsukuba (JP); Tomoyuki Shibuguchi, Tsukuba (JP)

(21) Appl. No.: 14/830,970

(22) Filed: Aug. 20, 2015

(30) Foreign Application Priority Data
Aug. 22, 2014 (JP) P2014-169189

(51) Int. Cl. C07D 498/04

(52) U.S. Cl. CPC C07D 498/04

(57) ABSTRACT

A compound represented by formula (I):

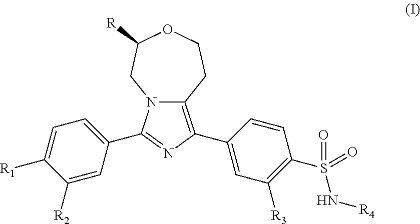

wherein R is a methyl group or the like, R₁ is a fluorine atom or the like, R₂ is a hydrogen atom or a fluorine atom, R₃ is a hydrogen atom, R₄ is an ethyl group or the like, or a pharmaceutically acceptable salt thereof.

### CROSS-REFERENCE TO RELATED APPLICATION

This application claims the benefit of Japanese application No. 2014-169189 filed on Aug. 22, 2014, the disclosure of which is herein incorporated by reference in its entirety.

### BACKGROUND OF THE INVENTION

1. Field of the Invention

The present invention relates to a tetrahydroimidazo[1,5-d][1,4]oxazepine compound having an antagonistic action against group II metabotropic glutamate receptor or a pharmaceutically acceptable salt thereof. The present invention also relates to a pharmaceutical composition comprising the compound as an active ingredient.

2. Related Background Art

Glutamic acid is known as one of principal excitatory neurotransmitters working for adjusting advanced functions of memory, learning and the like in a central nervous system of a mammal. Glutamate receptors are roughly classified into two types, that is, ionotropic glutamate receptors (iGlu receptors) and metabotropic glutamate receptors (mGlu receptors) coupled with G protein (see Non Patent Document 1).

The iGlu receptors are classified, on the basis of types of their agonists, into three types, that is, N-methyl-D-aspartate (NMDA) receptors, α-amino-3-hydroxy-5-methyl-4-isoxazolepropionic acid (AMPA) receptors and kainate receptors. On the other hand, the mGlu receptors have 8 subtypes (mGluR1 to 8) and are classified, on the basis of a signaling system to be conjugated and pharmacological characteristics, into group I (mGluR1, mGluR5), group II (mGluR2, mGluR3) and group III (mGluR4, mGluR6, mGluR7 and mGluR8). The group II and group III mGluRs are expressed as an autoreceptor or a heteroreceptor mainly at the nerve terminal, so as to suppress adenylate cyclase via Gi protein and regulate a specific K⁺ or Ca²⁺ channel activity (see Non Patent Document 2).

Antagonists against group II mGluRs, among these glutamate receptors, show an action to improve the cognitive function in animal models and also show an antidepressant action and an antianxiety action, and therefore, it is suggested that group II mGluR antagonists are effective as a novel cognitive function enhancer or antidepressant (see Non Patent Documents 3, 4 and 5).

### CITATION LIST

[Non-Patent Literature 1] Science, 258, 597-603, 1992
[Non-Patent Literature 2] Trends Pharmacol. Sci., 14, 13 (1993)
[Non-Patent Literature 3] Neuropharmacol., 46 (7), 907-917 (2004)
[Non-Patent Literature 4] Pharmacol. Therapeutics, 104(3), 233-244 (2004)
[Non-Patent Literature 5] Neuropharmacol., 66, 40-52 (2013)

### SUMMARY OF THE INVENTION

An object of the present invention is to provide a tetrahydroimidazo[1,5-d][1,4]oxazepine compound or a pharmaceutically acceptable salt thereof having an antagonistic action against group II metabotropic glutamate receptors, and a pharmaceutical composition comprising the same.

The present invention relate to [1] to [17] below:

[1]A compound represented by formula (I) or a pharmaceutically acceptable salt thereof:

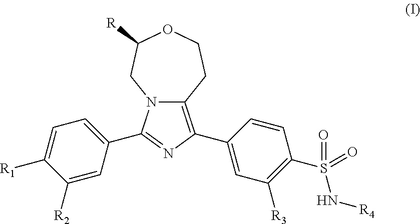

wherein

R is a methyl group or a fluoromethyl group,

R₁ is a fluorine atom, a methoxy group, an ethoxy group, a fluoromethyloxy group, a difluoromethyloxy group, or an oxetan-3-yloxy group,

R₂ is a hydrogen atom or a fluorine atom,

R₃ is a hydrogen atom,

R₄ is a methyl group, an ethyl group, an isopropyl group, a tert-butyl group, a cyclopropyl group, a cyclobutyl group, or a 1-methylcyclobutyl group.

[2]A compound selected from the following compounds or a pharmaceutically acceptable salt thereof:
(R)—N-isopropyl-4-(3-(4-methoxyphenyl)-6-methyl-5,6,8,9-tetrahydroimidazo[1,5-d][1,4]oxazepin-1-yl)benzenesulfonamide,
(R)—N-cyclobutyl-4-(3-(4-methoxyphenyl)-6-methyl-5,6,8,9-tetrahydroimidazo[1,5-d][1,4]oxazepin-1-yl)benzenesulfonamide,
(R)-4-(3-(4-fluorophenyl)-6-methyl-5,6,8,9-tetrahydroimidazo[1,5-d][1,4]oxazepin-1-yl)-N-isopropylbenzenesulfonamide, and
(S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide,
(R)—N-isopropyl-4-(3-(3-fluoro-4-methoxyphenyl)-6-methyl-5,6,8,9-tetrahydroimidazo[1,5-d][1,4]oxazepin-1-yl)benzenesulfonamide.
   [3] (R)—N-cyclobutyl-4-(3-(4-methoxyphenyl)-6-methyl-5,6,8,9-tetrahydroimidazo[1,5-d][1,4]oxazepin-1-yl)benzenesulfonamide represented by the following formula:

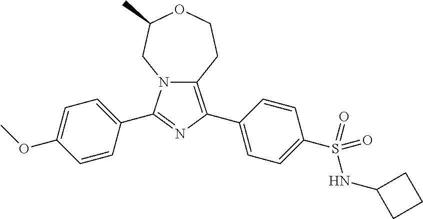 [SMILES: COc1ccc(-c2nc(-c3ccc(S(=O)(=O)NC4CCC4)cc3)c3n2C[C@@H](C)OCC3)cc1]

or a pharmaceutically acceptable salt thereof.
[4] (R)-4-(3-(4-fluorophenyl)-6-methyl-5,6,8,9-tetrahydroimidazo[1,5-d][1,4]oxazepin-1-yl)-N-isopropylbenzenesulfonamide represented by the following formula:

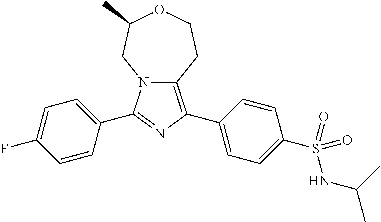 [SMILES: CC(C)NS(=O)(=O)c1ccc(-c2nc(-c3ccc(F)cc3)n3c2CCO[C@H](C)C3)cc1]

or a pharmaceutically acceptable salt thereof.
[5] (S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide represented by the following formula:

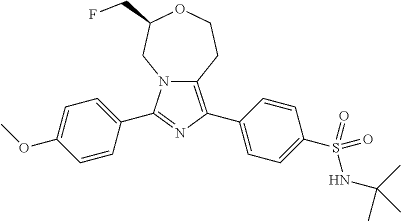 [SMILES: COc1ccc(-c2nc(-c3ccc(S(=O)(=O)NC(C)(C)C)cc3)c3n2C[C@@H](CF)OCC3)cc1]

or a pharmaceutically acceptable salt thereof.
[6]A pharmaceutical composition comprising the compound or a pharmaceutically acceptable salt thereof according to any of [1] to [5] and at least one pharmaceutically acceptable excipient.
[7] The pharmaceutical composition according to [6], for treatment of a disease or a symptom against which a group II metabotropic glutamate receptor antagonistic action is effective.
[8] The pharmaceutical composition according to [7], wherein the disease or the symptom is Alzheimer's disease.
[9]A method of treating a disease or a symptom against which a group II metabotropic glutamate receptor antagonistic action is effective, comprising administering the compound or a pharmaceutically acceptable salt thereof according to any of [1] to [5] to a subject in need thereof.
[10] The method according to [9], wherein the disease or the symptom is Alzheimer's disease.
[11] The compound or a pharmaceutically acceptable salt thereof according to any of [1] to [5], for use in a method of treating of a disease or a symptom against which a group II metabotropic glutamate receptor antagonistic action is effective.
[12] The compound or a pharmaceutically acceptable salt thereof according to [11], wherein the disease or the symptom is Alzheimer's disease.
[13] Use of the compound or a pharmaceutically acceptable salt thereof according to any of [1] to [5] for the manufacture of a pharmaceutical composition for treatment of a disease or a symptom against which a group II metabotropic glutamate receptor antagonistic action is effective.
[14] The use according to [13], wherein the disease or the symptom is Alzheimer's disease.
[15] The compound or a pharmaceutically acceptable salt thereof according to any of [1] to [5], for use as an active component of a pharmaceutical composition.
[16] The compound or a pharmaceutically acceptable salt thereof according to [15], wherein the pharmaceutical composition is a pharmaceutical composition for treatment of a disease or a symptom against which a group II metabotropic glutamate receptor antagonistic action is effective.
[17] The compound or a pharmaceutically acceptable salt thereof according to [16], wherein the disease or the symptom is Alzheimer's disease.

### Advantageous Effects of Invention

The compound of the present invention represented by formula (I) (hereinafter also referred to as the tetrahydroimidazo[1,5-d][1,4]oxazepine compound) or a pharmaceutically acceptable salt thereof shows an antagonistic action against group II metabotropic glutamate receptors. Therefore, the tetrahydroimidazo[1,5-d][1,4]oxazepine compound of the present invention or a pharmaceutically acceptable salt thereof has a potential use as a therapeutic agent for diseases or symptoms for which the antagonistic action against group II metabotropic glutamate receptors effectively works, such as Alzheimer's disease.

### DESCRIPTION OF THE PREFERRED EMBODIMENTS

Hereinafter, the meanings of signs, terms and the like used herein will be explained, and the present invention will be described in details.

Herein, a chemical formula of a compound may represent a given isomer for convenience, but a compound of the present invention includes isomers, such as all geometric isomers structurally formed from the compound, optical isomers based on asymmetric carbon, stereoisomers and tautomers, and the isomeric mixtures thereof. The compound is not limited to the formula given for convenience, and it may be any one of the isomers and mixtures. Accordingly, the compound of the present invention may have an asymmetric carbon atom in a molecule thereof and exist as an optically active substance and a racemic form. However, the present invention is not limited thereto, but it includes all cases. Incidentally, any one of isomers, racemic compounds and mixtures of isomers may show stronger activity than the other isomers. Furthermore, there may exist crystal polymorphisms, which also does not limit the present invention, and the compound may be any of single crystals or mixtures thereof, and may be a hydrate or a solvate as well as an anhydrate, all of which are included in the scope of the claims herein.

The present invention includes an isotopically-labeled compound of the compound of formula (I). The isotopically-labeled compound is equivalent to the compound of formula (I) except that one or more of atoms are replaced by atom(s) having an atomic mass or a mass number different from those usually found in nature. Examples of an isotope that can be incorporated into the compound of the present invention include isotopes of hydrogen, carbon, nitrogen, oxygen, fluorine, chlorine, phosphorus, sulfur and iodine, such as ²H, ³H, ¹¹C, ¹⁴C, ¹³N, ¹⁵O, ¹⁸F, ³²P, ³⁵S, ¹²³I and ¹²⁵I.

The compound of the present invention containing any of the aforementioned isotopes and/or another isotope, and a pharmaceutically acceptable derivative (such as a salt) thereof fall in the scope of the claims herein. The isotopically-labeled compound of the present invention, for example those into which radioactive isotopes such as ³H and/or ¹⁴C are incorporated, may be useful in drug and/or substrate tissue distribution assays. The isotopes ³H and ¹⁴C are regarded to be useful because these isotopes can be easily prepared and detected. The isotopes ¹¹C and ¹⁸F are regarded to be useful in PET (positron emission tomography), the isotope ¹²⁵I is regarded to be useful in SPECT (single photon emission computed tomography), and these isotopes are all useful in brain imaging. Replacement by a heavier isotope such as ²H causes, because of its higher metabolic stability, some types of advantages, in a treatment, of, for example, extension of half-life in vivo or reduction of a necessary dose, and therefore, is regarded useful under given circumstances. An isotopically-labeled compound of the compound of formula (I) of the present invention can be similarly prepared by using a readily available isotopically-labeled reagent instead of a nonisotopically-labeled reagent and by performing procedures disclosed in schemes and/or examples described below.

The tetrahydroimidazo[1,5-d][1,4]oxazepine compound of formula (I) of the present invention may be in the form of a pharmaceutically acceptable salt. Specific examples of the pharmaceutically acceptable salt include acid addition salts such as inorganic acid salts (such as a sulfate, a nitrate, a perchlorate, a phosphate, a carbonate, a bicarbonate, a hydrofluoride, a hydrochloride, a hydrobromide and a hydroiodide), organic carboxylates (such as an acetate, an oxalate, a maleate, a tartrate, a fumarate and a citrate), organic sulfonates (such as a methanesulfonate, a trifluoromethanesulfonate, an ethanesulfonate, a benzene sulfonate, a toluene sulfonate and a camphorsulfonate), and amino acid salts (such as an aspartate and a glutamate). Further, examples of the pharmaceutically acceptable salt include inorganic base salts such as alkali metal salts (such as a sodium salt and a potassium salt), alkaline earth metal salts (such as a calcium salt and a magnesium salt), aluminum salts, and ammonium salts.

An embodiment of the present invention is a compound represented by formula (I):

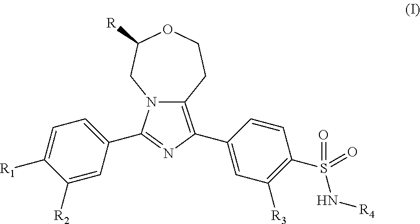

wherein R, R₁, R₂, R₃, and R₄ are the same as defined in the above [1],

or a pharmaceutically acceptable salt thereof.

More specifically, the tetrahydroimidazo[1,5-d][1,4]oxazepine compound or a pharmaceutically acceptable salt thereof according to the present invention is preferably a tetrahydroimidazo[1,5-d][1,4]oxazepine compound or a pharmaceutically acceptable salt thereof selected from the following compounds:
(R)—N-isopropyl-4-(3-(4-methoxyphenyl)-6-methyl-5,6,8,9-tetrahydroimidazo[1,5-d][1,4]oxazepin-1-yl)benzenesulfonamide,
(R)—N-cyclobutyl-4-(3-(4-methoxyphenyl)-6-methyl-5,6,8,9-tetrahydroimidazo[1,5-d][1,4]oxazepin-1-yl)benzenesulfonamide,
(R)-4-(3-(4-fluorophenyl)-6-methyl-5,6,8,9-tetrahydroimidazo[1,5-d][1,4]oxazepin-1-yl)-N-isopropylbenzenesulfonamide, and
(S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide.

Further preferable examples of the tetrahydroimidazo[1,5-d][1,4]oxazepine compound or a pharmaceutically acceptable salt thereof include:
(R)—N-cyclobutyl-4-(3-(4-methoxyphenyl)-6-methyl-5,6,8,9-tetrahydroimidazo[1,5-d][1,4]oxazepin-1-yl)benzenesulfonamide represented by the following formula:

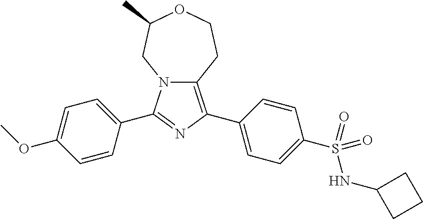 [SMILES: COc1ccc(-c2nc(-c3ccc(S(=O)(=O)NC4CCC4)cc3)c3n2C[C@@H](C)OCC3)cc1]

or a pharmaceutically acceptable salt thereof;
(R)-4-(3-(4-fluorophenyl)-6-methyl-5,6,8,9-tetrahydroimidazo[1,5-d][1,4]oxazepin-1-yl)-N-isopropylbenzenesulfonamide represented by the following formula:

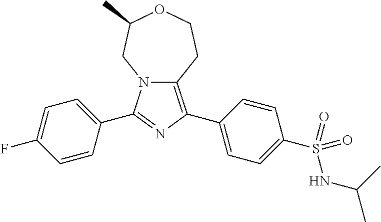 [SMILES: CC(C)NS(=O)(=O)c1ccc(-c2nc(-c3ccc(F)cc3)n3c2CCO[C@H](C)C3)cc1]

or a pharmaceutically acceptable salt thereof; and
(S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide represented by the following formula:

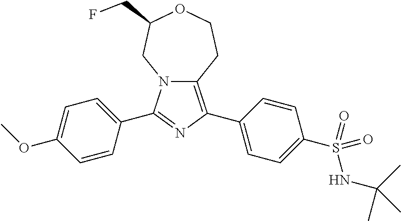 [SMILES: COc1ccc(-c2nc(-c3ccc(S(=O)(=O)NC(C)(C)C)cc3)c3n2C[C@@H](CF)OCC3)cc1]

or a pharmaceutically acceptable salt thereof.

Subsequently, a method for producing a compound of formula (I) according to the present invention (hereinafter referred to as compound (I). Compounds represented by other formulas are expressed in the same way.) or a pharmaceutically acceptable salt thereof will be described.

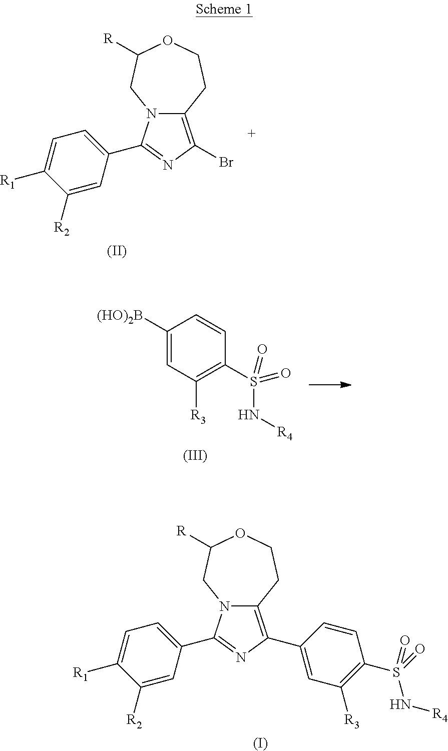

The compound (I) (wherein R, R₁, R₂, R₃ and R₄ represent the same as defined above) can be prepared in accordance with Scheme 1 by, for example, the Suzuki-Miyaura reaction of a compound (II) with a compound (III). The Suzuki-Miyaura reaction can be performed by heating the compound (II) and the compound (III) in a solvent in the presence of, for example, a palladium catalyst and a base, with a phosphorus ligand added if necessary. As the palladium catalyst, for example, tetrakis(triphenylphosphine)palladium (0), palladium (II) acetate, Pd₂DBA₃ or (A-taPhos)₂PdCl₂ can be used. As the base, for example, potassium phosphate, sodium hydroxide, potassium hydroxide, barium hydroxide, sodium carbonate or cesium carbonate can be used. Besides, as the phosphorus ligand, for example, triphenylphosphine, butyl di(1-adamantyl)phosphine or 2-dicyclohexylphosphino-2′,4′,6′-triisopropyl biphenyl can be used. The solvent used in the reaction is not especially limited as long as it is an inert solvent, and for example, THF, DME, DMF, 1,4-dioxane, water or a mixed solvent of these can be used. The reaction is accelerated by heating, but is generally performed at a temperature ranging from room temperature to the reflux temperature of the solution, and heating by microwaves can be employed as occasion demands.

When R₁ is, for example, an alkoxy group, the compound can be also produced by alkylating a compound, which is obtained by deprotecting a corresponding alcohol compound protected by MOM, benzyl, methyl or the like, with alkyl bromide, alkyl iodide, alkyl triflate or the like, in a solvent such as DMF or THF in the presence of a base such as potassium carbonate or cesium carbonate. This reaction is generally performed at a temperature ranging from room temperature to the reflux temperature of the solution.

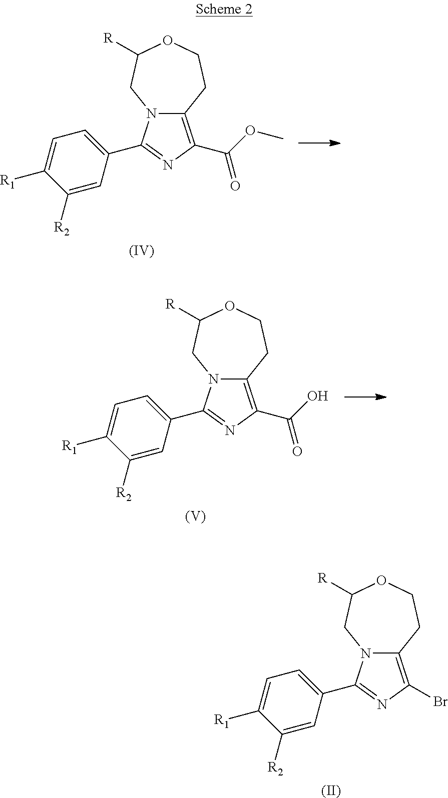

The compound (II) (wherein R, R₁ and R₂ represent the same as defined above) can be prepared in accordance with Scheme 2 by, for example, ester hydrolysis of a compound (IV) and decarboxylative bromination of a resulting compound (V). A solvent used in the ester hydrolysis of the compound (IV) is not especially limited as long as it is an inert solvent, and for example, methanol, ethanol, THF or a hydrous solvent thereof can be used. Besides, as a base, for example, sodium hydroxide or potassium hydroxide can be used. This reaction is accelerated by heating, but is generally performed at a temperature ranging from room temperature to the reflux temperature of the solution. A solvent used in the decarboxylative bromination of the compound (V) is not especially limited, and for example, DMF, ethanol or a mixed solvent of DMF and ethanol can be used. Furthermore, a bromine source can be, for example, NBS. If potassium carbonate or the like is used as the base, the reaction is accelerated, and the reaction is generally performed at a temperature ranging from room temperature to the reflux temperature of the solution.

When R₁ is, for example, an alkoxy group, the compound can be also produced by alkylating a compound, which is obtained by deprotecting a corresponding alcohol compound protected by MOM, benzyl, methyl or the like, with alkyl bromide, alkyl iodide, alkyl triflate or the like in a solvent such as DMF or THF in the presence of a base such as potassium carbonate or cesium carbonate. This reaction is generally performed at a temperature ranging from room temperature to the reflux temperature of the solution.

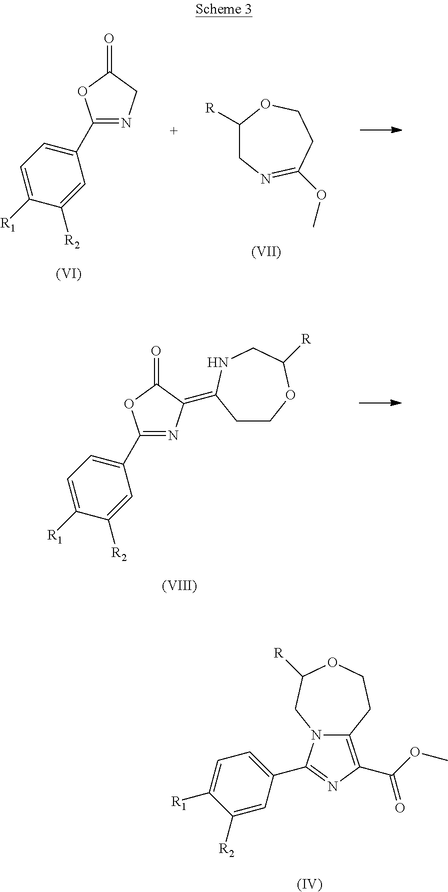

The compound (IV) (wherein R, R₁ and R₂ represent the same as defined above) can be prepared in accordance with Scheme 3 by, for example, condensing a compound (VI) with a compound (VII) and treating a resulting compound (VIII) with a base. A solvent used in the condensation of the compounds (VI) and (VII) is not especially limited as long as it is an inert solvent, and for example, toluene, THF, DME or a mixed solvent of these can be used. The reaction is accelerated by heating, but is generally performed at a temperature ranging from room temperature to the reflux temperature of the solution, and heating with microwaves can be employed as occasion demands. A solvent used in the treatment of the compound (VIII) with a base is not especially limited as long as it is an inert solvent, and for example, methanol can be used. The base can be, for example, sodium methoxide. The reaction is accelerated by heating, but is generally performed at a temperature ranging from room temperature to the reflux temperature of the solution, and heating with microwaves can be employed as occasion demands.

When R₁ is, for example, an alkoxy group, the compound can be also produced by alkylating a compound, which is obtained by deprotecting a corresponding alcohol compound protected by MOM, benzyl, methyl or the like, with alkyl bromide, alkyl iodide, alkyl triflate or the like in a solvent such as DMF or THF in the presence of a base such as potassium carbonate or cesium carbonate. This reaction is generally performed at a temperature ranging from room temperature to the reflux temperature of the solution.

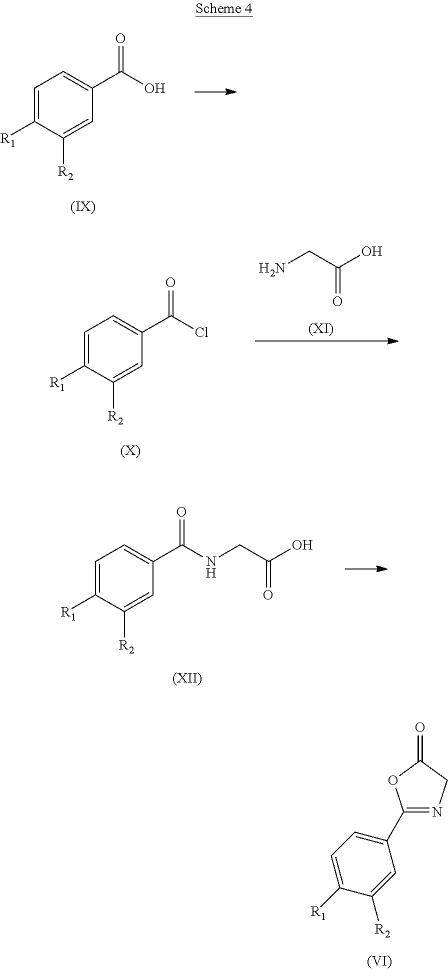

The compound (VI) (wherein R₁ and R₂ represent the same as defined above) can be prepared in accordance with Scheme 4 by, for example, acid chloridization of a compound (IX), amidation of a resulting compound (X) and a compound (XI) under basic conditions, and cyclization of a resulting compound (XII). A solvent used in the acid chloridization of the compound (IX) is not especially limited as long as it is an inert solvent, and for example, toluene or DCM can be used. Furthermore, for example, oxalyl chloride or thionyl chloride can be used for the reaction, and the reaction is accelerated by addition of DMF. The reaction is accelerated by heating, but is generally performed at a temperature ranging from an ice cooling temperature to the reflux temperature of the solution. A solvent used in the amidation of the compounds (X) and (XI) is not especially limited as long as it is an inert solvent, and for example, toluene, THF, DCM, water or a mixed solvent of these can be used. Furthermore, as a base, for example, sodium hydroxide or potassium hydroxide can be used. This reaction is generally performed at a temperature ranging from an ice cooling temperature to the reflux temperature of the solution. A solvent used in the cyclization of the compound (XII) is not especially limited as long as it is an inert solvent, and for example, toluene or THF can be used. Besides, methyl chloroformate, isopropyl chloroformate, DCC or the like can be used for the cyclization. This reaction is generally performed at a temperature ranging from −78° C. to the reflux temperature of the solution.

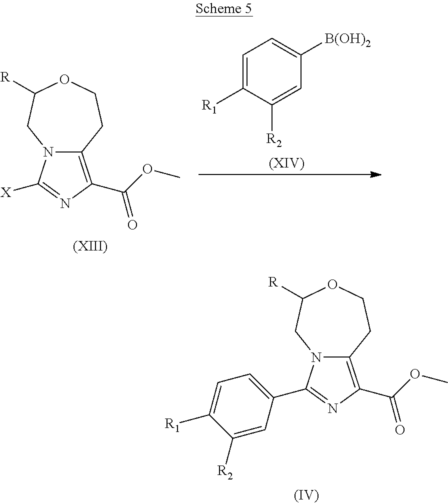

The compound (IV) (wherein R, R₁ and R₂ represent the same as defined above) can be prepared also in accordance with Scheme 5 by, for example, the Suzuki-Miyaura reaction of a compound (XIII) (wherein X is halogen) and a compound (XIV). The Suzuki-Miyaura reaction can be performed by heating the compound (XIII) and the compound (XIV) in a solvent in the presence of, for example, a palladium catalyst and a base, with a phosphorus ligand added if necessary. As the palladium catalyst, for example, tetrakis(triphenylphosphine)palladium (0), palladium (II) acetate, Pd₂DBA₃ or (A-taPhos)₂PdCl₂ can be used. As the base, for example, potassium phosphate, sodium hydroxide, potassium hydroxide, barium hydroxide, sodium carbonate or cesium carbonate can be used. Besides, as the phosphorus ligand, for example, triphenylphosphine, butyl di(1-adamantyl)phosphine or 2-dicyclohexylphosphino-2′,4′,6′-triisopropyl biphenyl can be used. The solvent used in the reaction is not especially limited as long as it is an inert solvent, and for example, THF, DME, DMF, 1,4-dioxane or benzene can be used. The reaction is accelerated by heating, but is generally performed at a temperature ranging from room temperature to the reflux temperature of the solution, and heating by

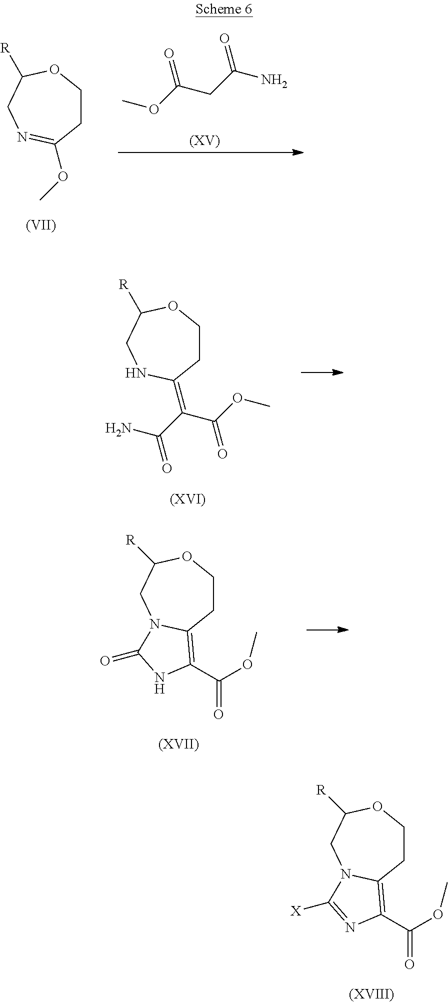

The compound (XIII) (wherein R is the same as defined above and X is halogen) can be prepared in accordance with Scheme 6 by, for example, condensation of the compound (VII) with a compound (XV), a Hofmann rearrangement reaction of a resulting compound (XVI), and halogenation of a resulting compound (XVII). A solvent used in the condensation of the compounds (VII) and (XV) is not especially limited as long as it is an inert solvent, and for example, toluene, THF, DMF, DME or a mixed solvent of these can be used. The reaction is accelerated by heating, but is generally performed at a temperature ranging from room temperature to the reflux temperature of the solution, and heating with microwaves can be employed as occasion demands. A solvent used in the rearrangement reaction of the compound (XVI) is not especially limited as long as it is an inert solvent, and for example, toluene, THF, DME or a mixed solvent of these can be used. Furthermore, iodobenzene diacetate or the like can be used in the reaction. The reaction is generally performed at a temperature ranging from room temperature to the reflux temperature of the solution. A solvent used in the halogenation of the compound (XVII) is not especially limited as long as it is an inert solvent, and for example, toluene can be used. Furthermore, phosphorus oxychloride or phosphorus oxybromide can be used in the reaction. The reaction is accelerated by heating, but is generally performed at a temperature ranging from room temperature to the reflux temperature of the solution.

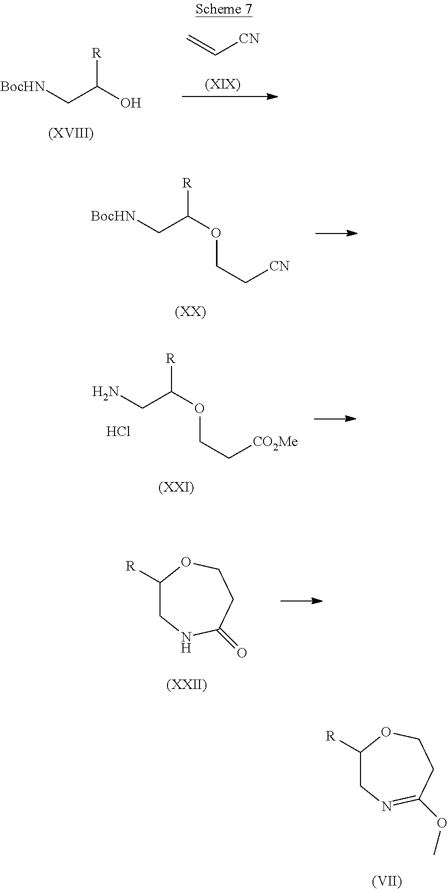

The compound (VII) (wherein R is the same as defined above) can be prepared in accordance with Scheme 7 by, for example, four steps of a 1,4-addition reaction of a compound (XVIII) and a compound (XIX), alcoholysis of a resulting compound (XX) under acidic conditions, cyclization of a resulting compound (XXI) under basic conditions, and O-alkylation of a resulting compound (XXII). In the 1,4-addition reaction of the compound (XVIII), the compound (XIX) can be used as a solvent. As a base, DBU, TEA, DIPEA or the like can be used. This reaction is generally performed at a temperature ranging from an ice cooling temperature to the reflux temperature of the solution. A solvent used in the alcoholysis of the compound (XX) is not especially limited as long as it is an inert solvent, and for example, 1,4-dioxane can be used. As an acid, hydrogen chloride or the like can be used. This reaction is accelerated by heating, but is generally performed at a temperature ranging from room temperature to the reflux temperature of the solution. A solvent used in the cyclization of the compound (XXI) is not especially limited as long as it is an inert solvent, and for example, methanol or the like can be used. As a base, DBU, TEA, potassium carbonate or cesium carbonate can be used. This reaction is accelerated by heating, but is generally performed at a temperature ranging from room temperature to the reflux temperature of the solution. A solvent used in the O-alkylation of the compound (XXII) is not especially limited as long as it is an inert solvent, and for example, DCM or toluene can be used. As an alkylating agent, trimethyloxonium tetrafluoroborate, dimethyl sulfate or the like can be used. This reaction is generally performed at a temperature ranging from an ice cooling temperature to the reflux temperature of the solution.

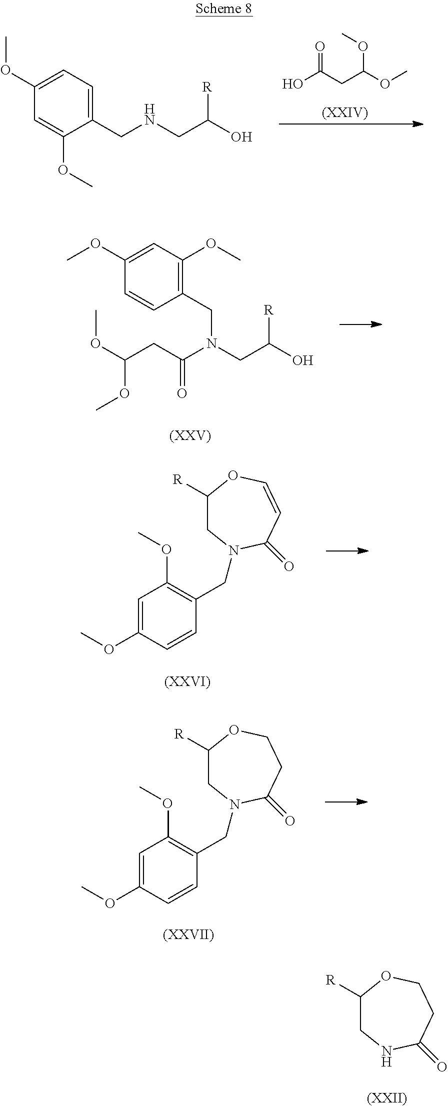

The compound (XXII) (wherein R is the same as defined above) can also be prepared in accordance with Scheme 8 by, for example, four steps of dehydrative condensation of a compound (XXIII) with a compound (XXIV), cyclization of a resulting compound (XXV) performed under acidic conditions, hydrogenation of a resulting compound (XXVI), and deprotection of a resulting compound (XXVII). A solvent used in the dehydrative condensation of the compound (XXIII) with the compound (XXIV) is not especially limited as long as it is an inert solvent, and for example, THF, DMF or DCM can be used. Besides, a condensation agent can be DCC, EDC, HOBT, HATU, HBTU or a combination of any of these. Furthermore, DIPEA, TEA or the like can be used as an additive in the reaction. This reaction is generally performed at a temperature ranging from an ice cooling temperature to the reflux temperature of the solution. A solvent used in the cyclization of the compound (XXV) is not especially limited as long as it is an inert solvent, and for example, THF, acetonitrile, toluene or xylene can be used. Besides, an acid can be, for example, PTS or PPTS. The reaction is accelerated by heating, but is generally performed at a temperature ranging from room temperature to the reflux temperature of the solution. A solvent used in the hydrogenation of the compound (XXVI) is not especially limited as long as it is an inert solvent, and for example, methanol, ethanol or THF can be used. As a catalyst, palladium/carbon, palladium hydroxide/carbon, platinum oxide or the like can be used. This reaction is generally performed at a temperature ranging from room temperature to the reflux temperature of the solution. The deprotection of the compound (XXVII) can be performed, for example, in a solvent such as TFA. As an additive, for example, a scavenger such as a triethyl silane can be used. This reaction is accelerated by heating, but is generally performed at a temperature ranging from room temperature to the reflux temperature of the solution.

The compound (I) of the present invention thus obtained can be prepared into a pharmaceutically acceptable salt by a conventional method as occasion demands. The preparation method can be an appropriate combination of, for example, methods conventionally employed in the field of synthetic organic chemistry. A specific example of the method includes neutralization titration of a solution of the free form of the present compound with an acid solution. Furthermore, the compound (I) of the present invention can be changed into a solvate by a known solvate forming reaction as occasion demands.

Representative examples of the method for producing the compound (I) have been described so far, and material compounds and various reagents used in the production method for the compound (I) may be in the form of a salt or a hydrate, and are different depending upon starting materials, solvents to be used and the like, and hence are not especially limited as long as the reactions are not retarded. Also the solvents to be used differ depending upon the starting materials, reagents and the like, and needless to say, are not especially limited as long as they do not retard the reactions and they dissolve starting materials to some extent. When the compound (I) is obtained in the form of a free form, it can be changed, by a conventional method, into the form of a salt that can be formed by the compound (I). Similarly, when the compound (I) is obtained in the form of a salt of the compound (I), it can be changed, by a conventional method, into a free form of the compound (I). Furthermore, various isomers (such as a geometric isomer, an optical isomer based on asymmetric carbon, a stereoisomer and a tautomer) obtained as the compound (I) can be purified and isolated by general separation means such as recrystallization, a diastereomeric salt formation method, enzymatic resolution, and various types of chromatography (including thin layer chromatography, column chromatography and gas chromatography).

A term “composition” used herein includes a product that contains a specific ingredient in a particular amount, and any product directly or indirectly prepared by a combination of particular ingredients in particular amounts. Such a term used in regard to a pharmaceutical composition is used to intend to include: a product containing an active ingredient and an inactive ingredient forming a carrier; and all products directly or indirectly prepared by combination, complexation or aggregation of any two or more ingredients, or dissociation, another type of reaction, or interaction of one or more ingredients. Accordingly, the pharmaceutical composition of the present invention includes all compositions prepared by mixing the tetrahydroimidazo[1,5-d][1,4]oxazepine compound of the present invention with any of pharmaceutically acceptable carriers. The term “pharmaceutically acceptable” means that a carrier, a diluent or an excipient should be compatible with other ingredients of the formulation and should not be harmful to those who take the composition.

The compounds of the present invention mostly have, as binding ability to the group II metabotropic glutamate receptors, an IC50 value of 100 nM or less, and have an IC50 value of preferably 30 nM or less and more preferably 10 nM or less.

The tetrahydroimidazo[1,5-d][1,4]oxazepine compound of the present invention or a pharmaceutically acceptable salt thereof has an antagonistic action against the group II metabotropic glutamate receptors. Accordingly, it is applicable as a therapeutic agent for diseases in which the antagonistic action against the group II metabotropic glutamate receptors effectively works. Examples of the diseases in which the antagonistic action against the group II metabotropic glutamate receptors effectively works include Alzheimer's disease.

The tetrahydroimidazo[1,5-d][1,4]oxazepine compound of the present invention or a pharmaceutically acceptable salt thereof can be formulated by a general method, and the dosage form can be, for example, an oral formulation (such as a tablet, a granule, a powder, a capsule or a syrup), an injection formulation (for intravenous administration, intramuscular administration, subcutaneous administration, intraperitoneal administration or the like), or an external formulation (such as a transdermal absorbable drug (including an ointment, a patch and the like), an eye dropper, nasal drops or a suppository).

For producing an oral solid formulation, an excipient, a binder, a disintegrator, a lubricant, a colorant and the like can be added, if necessary, to the tetrahydroimidazo[1,5-d][1,4]oxazepine compound or a pharmaceutically acceptable salt thereof of the present invention, and the resulting mixture can be prepared by a conventional method into tablets, granules, powders or capsules. Furthermore, the tablets, granules, powders or capsules can be coated with a film if necessary.

Examples of the excipient include lactose, corn starch and crystalline cellulose, examples of the binder include hydroxypropyl cellulose and hydroxypropylmethyl cellulose, examples of the disintegrator include carboxymethylcellulose calcium and croscarmellose sodium, examples of the lubricant include magnesium stearate, an example of the colorant includes titanium oxide, and examples of a coating agent include hydroxypropyl cellulose, hydroxypropylmethyl cellulose and methyl cellulose, and it goes without saying that these ingredients are not limited to the aforementioned examples.

These solid preparations such as tablets, capsules, granules and powders may usually include an arbitrary amount of a tetrahydroimidazo[1,5-d][1,4]oxazepine compound or a pharmaceutically acceptable salt thereof according to the present invention or a solvate thereof, as long as the solid preparations show a pharmacological effect in such a manner that they can be used as medicines.

For producing an injection formulation (for intravenous administration, intramuscular administration, subcutaneous administration, intraperitoneal administration or the like), a pH adjuster, a buffer, a suspending agent, a solubilizing agent, an antioxidant, a preservative (an antiseptic agent), a tonicity adjusting agent and the like are added, if necessary, to the tetrahydroimidazo[1,5-d][1,4]oxazepine compound or a pharmaceutically acceptable salt thereof of the present invention, and the resulting mixture can be prepared into an injection formulation by a conventional method. Furthermore, the resultant can be freeze-dried to be used as a lyophilized product to be dissolved before use.

Examples of the pH adjuster and the buffer include organic acids, inorganic acids and/or pharmaceutically acceptable salts thereof, examples of the suspending agent include methyl cellulose, examples of the solubilizing agent include Polysorbate 80, an example of the antioxidant includes α-tocopherol, examples of the preservative include methyl paraoxybenzoate and ethyl paraoxybenzoate, and examples of the tonicity adjusting agent include glucose, and it goes without saying that these ingredients are not limited to the aforementioned examples.

Such an injection formulation may usually include an arbitrary amount of a tetrahydroimidazo[1,5-d][1,4]oxazepine compound or a pharmaceutically acceptable salt thereof according to the present invention or a solvate thereof, as long as the solid preparations show a pharmacological effect in such a manner that they can be used as medicines.

For producing an external formulation, a base material is added to the tetrahydroimidazo[1,5-d][1,4]oxazepine compound or a pharmaceutically acceptable salt thereof of the present invention, and if necessary, for example, a preservative, a stabilizer, a pH adjuster, an antioxidant, a colorant and the like described above are further added thereto, and the resulting mixture is prepared by a conventional method into, for example, a transdermal absorbable drug (such as an ointment or a patch), an eye dropper, nasal drops or a suppository.

As the base material to be used, various materials usually used for, for example, medicines, quasi-drugs and cosmetics can be used. Specific examples of the material include animal and vegetable oils, mineral oils, ester oils, waxes, emulsifiers, higher alcohols, fatty acids, silicone oils,

surfactants, phospholipids, alcohols, polyalcohols, water soluble polymers, clay minerals and purified water.

These external preparations may include an arbitrary amount of a tetrahydroimidazo[1,5-d][1,4]oxazepine compound or a pharmaceutically acceptable salt thereof according to the present invention or a solvate thereof, as long as the external preparations show a pharmacological effect in such a manner that they can be used as medicines.

A dosage of the tetrahydroimidazo[1,5-d][1,4]oxazepine compound of the present invention or a pharmaceutically acceptable salt thereof depends upon the level of symptom severity, the patient's age, sex and weight, the administration form and the kind of salt, a specific kind of disease and the like, and in an adult patient, it is administered, once or dividedly several times per day, at a dose for oral administration of generally approximately 30 g to 10 g, preferably 100 μg to 5 g and more preferably 100 μg to 1 g, or a dose for injection administration of generally approximately 30 g to 1 g, preferably 100 g to 500 mg, and more preferably 100 μg to 300 mg.

The compound of the present invention can be used as a chemical probe for capturing a target protein of a biologically active low molecular weight compound. Specifically, the compound of the present invention can be transformed into an affinity chromatography probe, a photo-affinity probe or the like by introducing a labeling group, a linker or the like into a portion other than a structural portion indispensable to activity expression of the compound by a method described in J. Mass Spectrum. Soc. Jpn. Vol. 51, No. 5, 2003, p. 492-498, WO2007/139149 or the like.

Examples of the labeling group, the linker or the like used in such a chemical probe include groups belonging to the following groups (1) to (5):

(1) protein labeling groups such as photoaffinity labeling groups (such as a benzoyl group, a benzophenone group, an azido group, a carbonyl azide group, a diaziridine group, an enone group, a diazo group and a nitro group), and chemical affinity groups (such as a ketone group in which an alpha carbon atom is substituted with a halogen atom, a carbamoyl group, an ester group, an alkylthio group, a Michael receptor of α,β-unsaturated ketone, ester or the like, and an oxirane group);

(2) cleavable linkers such as —S—S—, —O—Si—O—, a monosaccharide (such as a glucose group or a galactose group) and a disaccharide (such as lactose), and oligopeptide linkers that can be cleaved by an enzyme reaction;

(3) fishing tag groups such as biotin and a 3-(4,4-difluoro-5,7-dimethyl-4H-3a,4a-diaza-4-bora-s-indacen-3-yl)propionyl group;

(4) radioactive labeling groups such as ¹²⁵I, ³²P, ³H and ¹⁴C; fluorescence labeling groups such as fluorescein, rhodamine, dansyl, umbelliferone, 7-nitrofurazanyl, and a 3-(4,4-difluoro-5,7-dimethyl-4H-3a,4a-diaza-4-bora-s-indacen-3-yl) propionyl group; chemiluminescent groups such as lumiferin and luminol; and detectable markers such as heavy metal ions such as lanthanoid metal ions and radium ions; and

(5) groups to be bonded to a solid phase carrier such as glass beads, a glass bed, a microtiter plate, agarose beads, an agarose bed, polystyrene beads, a polystyrene bed, nylon beads and a nylon bed.

A probe prepared by introducing, into the compound of the present invention, a labeling group or the like selected from the above-described groups (1) to (5) by the method described in any of the aforementioned literatures or the like can be used as a chemical probe for identifying a marker protein useful for research of a novel potential drug target or the like.

Hereinafter, the present invention will be described in detail with reference to Examples, Production Examples, and Test Examples. However, the present invention is not limited to them. In addition, abbreviations used in Examples are commonly used abbreviations well known to the person skilled in the art, and some of the abbreviations will be described below.

(A-taPhos)₂PdCl₂: bis(di-tert-butyl(4-dimethylaminophenyl)phosphine)dichloropalladium(II)
DBN: 1,5-diazabicyclo[4.3.0]non-5-ene
DBU: 1,8-diazabicyclo[5.4.0]undec-7-ene
DCM: dichloromethane
DIPEA: diisopropylethylamine
DME: 1,2-dimethoxyethane

### DMF: N,N-dimethylformamide

EDC: 1-ethyl-3-(3-dimethylaminopropyl)carbodiimide hydrochloride
HFIP: hexafluoroisopropanol
HOBT: 1-hydroxybenzotriazole

### NBS: N-bromosuccinimide

### NMM: N-methylmorpholine

Pd(dppf)Cl₂.CH₂Cl₂: (1,1′-bis(diphenylphosphino)ferrocene-dichloropalladium-dichloromethane complex
PTS: paratoluenesulfonic acid
PPTS: pyridinium paratoluenesulfonate
TEA: triethylamine
TFA: trifluoroacetic acid
THF: tetrahydrofuran
¹H-NMR: proton nuclear magnetic resonance spectrometry

Chemical shifts of proton nuclear magnetic resonance spectra are recorded in the unit of 6 (ppm) with respect to tetramethylsilane and coupling constants are recorded in the unit of Herz (Hz). Patterns include: s: singlet, d: doublet, t: triplet, q: quartet, quin: quintet, and br: broad.

The term “room temperature” in Examples and Production Examples described below usually stands for a temperature in the range of about 10° C. to 35° C. The symbol “%” denotes % by weight, unless otherwise described.

The chemical names of compounds in Examples and Production Examples were determined based on their chemical structures with reference to “E-Notebook”, version 12 (PerkinElmer Inc.).

### Production Example 1

### Synthesis of (R)-5-methoxy-2-methyl-2,3,6,7-tetrahydro-1,4-oxazepine

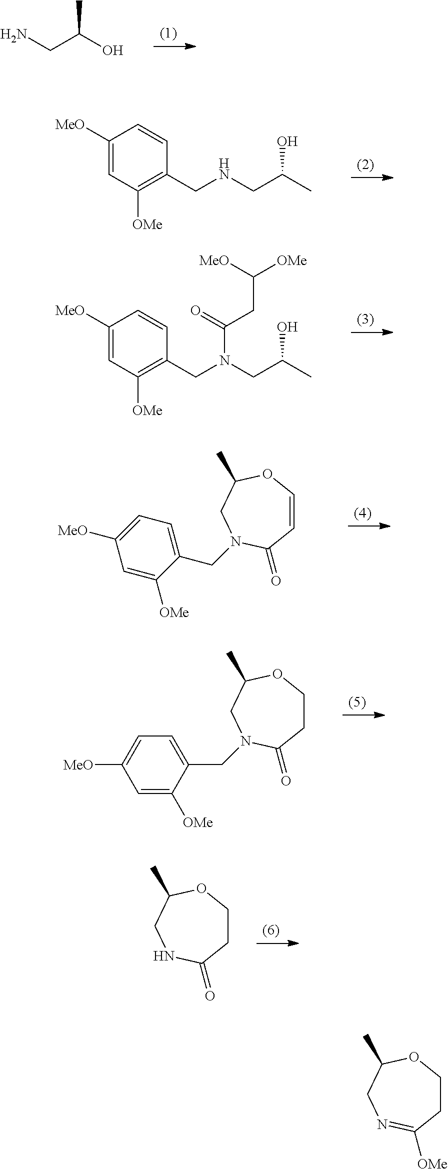

### (1) Synthesis of (R)-1-((2,4-dimethoxybenzyl)amino)propan-2-ol

2,4-Dimethoxybenzaldehyde (CAS No. 613-45-65; 55.8 g, 336 mmol) was added to a solution of (R)-(−)-1-amino-2-propanol (CAS No. 2799-16-8; 24.0 g, 320 mmol) and acetic acid (40.2 mL, 703 mmol) in THF (440 mL) at room temperature, and the mixture was stirred at room temperature for 1 hour. Sodium triacetoxyborohydride (102 g, 479 mmol) was added to the reaction liquid at room temperature, and the mixture was stirred for 18 hours. The solvent was concentrated under reduced pressure after the reaction. A 5 N aqueous sodium hydroxide solution (100 mL) and ethyl acetate (500 mL) were added to the resultant residue to separate the organic layer. Chloroform (300 mL) was added to the resultant water layer to separate the organic layer. The resultant organic layers were combined, and the resultant was washed with a saturated aqueous sodium chloride solution and then dried over anhydrous magnesium sulfate. The drying agent was filtered off, and then the solvent was evaporated under reduced pressure. The resultant residue was filtered through NH silica gel (ethyl acetate) for purification to obtain a crude title compound (72 g).

¹H-NMR (400 MHz, CDCl₃) δ (ppm): 1.13 (d, J=6.3 Hz, 3H), 2.34 (dd, J=9.4, 12.1 Hz, 1H), 2.68 (dd, J=3.1, 12.1 Hz, 1H), 3.72 (d, J=2.0 Hz, 2H), 3.75-3.79 (m, 1H), 3.80 (s, 3H), 3.82 (s, 3H), 6.39-6.48 (m, 2H), 7.10 (d, J=8.2 Hz, 1H).

### (2) Synthesis of (R)—N-(2,4-dimethoxybenzyl)-N-(2-hydroxypropyl)-3,3-dimethoxypropanamide

DIPEA (173 mL, 995 mmol) was added to a solution of the compound obtained in Production Example 1-(1) (74.7 g, 332 mmol), 3,3-dimethoxypropionic acid (CAS No. 6191-98-6; 38.5 g, 287 mmol), EDC (95 g, 497 mmol), and HOBT (67.2 g, 497 mmol) in DMF (500 mL) at room temperature, and the mixture was stirred for 14 hours. Water (1 L) and ethyl acetate (1 L) were added to the reaction mixture to separate the organic layer. The resultant organic layer was washed with water (1 L) and a saturated aqueous sodium chloride solution, then dried over anhydrous magnesium sulfate, the drying agent was filtered off, and the solvent was evaporated under reduced pressure. The resultant residue was purified by NH-silica gel column chromatography (n-heptane/ethyl acetate) to obtain a title compound (61 g, 179 mmol).

ESI-MS m/z 342 [M+H]⁺

### (3) Synthesis of (R)-4-(2,4-dimethoxybenzyl)-2-methyl-3,4-dihydro-1,4-oxazepin-5(2H)-one

PPTS (19.7 g, 78.4 mmol) was added to a solution of the compound obtained in Production Example 1-(2) (53.5 g, 157 mmol) in toluene (900 mL) at room temperature, and then the mixture was heated under reflux for 7 hours. The reaction mixture was cooled to room temperature and then a saturated aqueous sodium bicarbonate solution and ethyl acetate were added to separate the organic layer. The resultant organic layer was washed with a saturated aqueous sodium chloride solution and then dried over anhydrous magnesium sulfate, the drying agent was filtered off, and then the solvent was evaporated under reduced pressure. The resultant residue was purified by silica gel column chromatography (n-heptane/ethyl acetate) to obtain a title compound (30.5 g, 110 mmol).

¹H-NMR (400 MHz, CDCl₃) δ (ppm): 1.19 (d, J=6.6 Hz, 3H), 3.39-3.44 (m, 2H), 3.80 (s, 3H), 3.82 (s, 3H), 4.03-4.11 (m, 1H), 4.44 (d, J=14.5 Hz, 1H), 4.73 (d, J=14.5 Hz, 1H), 5.08 (d, J=8.2 Hz, 1H), 6.43-6.48 (m, 3H), 7.24 (d, J=9.0 Hz, 1H).

### (4) Synthesis of (R)-4-(2,4-dimethoxybenzyl)-2-methyl-1,4-oxazepan-5-one

20% Palladium hydroxide/carbon (3 g, including 50% water content) was added to a solution of the compound obtained in Production Example 1-(3) (30.5 g, 110 mmol) in methanol (500 mL) at room temperature, and the mixture was stirred under hydrogen atmosphere at 40° C. for 18 hours. The reaction mixture was cooled to room temperature and then was filtered through Celite (trademark), and the filtrate was concentrated under reduced pressure. The resultant residue was purified by silica gel column chromatography (ethyl acetate) to obtain a title compound (29.1 g, 104 mmol).

¹H-NMR (400 MHz, CDCl₃) δ (ppm): 1.05 (d, J=6.6 Hz, 3H), 2.60 (dd, J=5.1, 15.6 Hz, 1H), 2.92 (ddd, J=2.2, 11.0, 15.4 Hz, 1H), 3.20 (d, J=15.2 Hz, 1H), 3.29-3.38 (m, 1H), 3.40-3.50 (m, 1H), 3.56-3.66 (m, 1H), 3.81 (s, 3H), 3.82 (s, 3H), 3.96 (ddd, J=2.3, 5.5, 12.5 Hz, 1H), 4.37 (d, J=14.5 Hz, 1H), 4.70 (d, J=14.5 Hz, 1H), 6.43-6.48 (m, 2H), 7.21 (d, J=8.6 Hz, 1H).

### (5) Synthesis of (R)-2-methyl-1,4-oxazepan-5-one

Triethylsilane (26.2 mL, 164 mmol) was added to a solution of the compound obtained in Production Example 1-(4) (30.5 g, 110 mmol) in TFA (150 mL) at room temperature, and the mixture was stirred at 60° C. for 3 hours. The reaction mixture was cooled to room temperature and then concentrated under reduced pressure. The resultant residue was purified by silica gel column chromatography (ethyl acetate/methanol) to obtain a title compound (12.3 g, 95 mmol).

¹H-NMR (400 MHz, CDCl₃) δ (ppm): 1.19 (d, J=6.3 Hz, 3H), 2.48-2.58 (m, 1H), 2.89 (ddd, J=2.5, 10.9, 15.4 Hz, 1H), 3.03 (ddd, J=0.9, 7.6, 15.3 Hz, 1H), 3.35 (ddd, J=3.9, 8.4, 15.4 Hz, 1H), 3.57-3.76 (m, 2H), 4.01 (ddd, J=2.5, 5.3, 12.7 Hz, 1H), 5.85-6.07 (m, 1H).

### (6) Synthesis of (R)-5-methoxy-2-methyl-2,3,6,7-tetrahydro-1,4-oxazepine

Trimethyloxonium tetrafluoroborate (16.8 g, 114 mmol) was added to a solution of the compound obtained in Production Example 1-(5) (13.4 g, 103 mmol) in DCM (500 mL) at room temperature, and the mixture was stirred for 18 hours. A saturated aqueous sodium bicarbonate solution was added to the reaction mixture, and the organic layer was separated. DCM was added to the resultant water layer, and the organic layer was separated. The resultant organic layers were combined, the resultant was washed with a saturated aqueous sodium chloride solution, then the resultant was dried over anhydrous magnesium sulfate, and then the drying agent was filtered off and the solvent was evaporated under reduced pressure to obtain a title compound (13.7 g, 96 mmol).

¹H-NMR (400 MHz, CDCl₃) δ (ppm): 1.19 (d, J=6.4 Hz, 3H), 2.42 (ddd, J=1.2, 4.5, 15.6 Hz, 1H), 2.81-2.92 (m, 1H), 3.33-3.42 (m, 1H), 3.47-3.59 (m, 3H), 3.61 (s, 3H), 3.85-3.93 (m, 1H).

### Production Example 2

### Synthesis of (R)-2-methyl-1,4-oxazepan-5-one

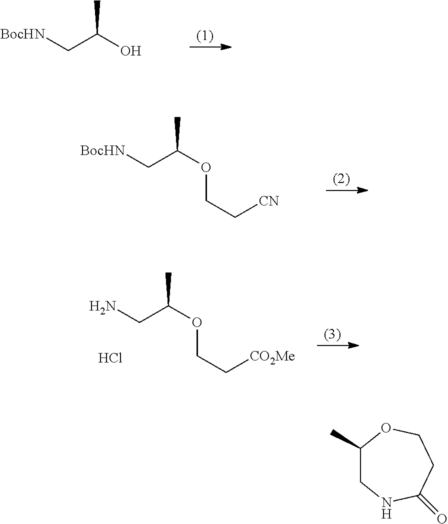

### (1) Synthesis of (R)-tert-butyl(2-(2-cyanoethoxy)propyl)carbamate

DBU (27.3 mL, 182 mmol) was added to a solution of (R)-tert-butyl(2-hydroxypropyl)carbamate (CAS No. 119768-44-4; 71.0 g, 405 mmol) in acrylonitrile (400 mL) at room temperature, and the mixture was stirred at the same temperature for 5 hours. Acetic acid (10.4 mL, 182 mmol) was added to the reaction mixture, and the mixture was concentrated under reduced pressure. The resultant residue was purified by silica gel column chromatography (n-heptane/ethyl acetate) to obtain a title compound (63.1 g, 276 mmol).

¹H-NMR (400 MHz, CDCl₃) δ (ppm): 1.10-1.20 (m, 3H), 1.45 (s, 9H), 2.59 (dd, J=6.3, 6.3 Hz, 2H), 2.96-3.11 (m, 1H), 3.23-3.41 (m, 1H), 3.52-3.66 (m, 1H), 3.61 (td, J=6.3, 9.2 Hz, 1H), 3.75 (td, J=6.3, 9.2 Hz, 1H), 4.88 (brs, 1H).

### (2) Synthesis of (R)-methyl 3-((1-aminopropan-2-yl)oxy)propanoate hydrochloride

The compound obtained in Production Example 2-(1) (63.1 g, 276 mmol) was dissolved in a 4 M hydrogen chloride/i 1,4-dioxane solution (691 mL) and a 5 to 10% hydrogen chloride/methanol solution (140 mL), and the mixture was stirred at 50° C. for 3 hours. A 4 M hydrogen chloride/1,4-dioxane solution (311 mL) was further added to the reaction mixture, the mixture was stirred at 50° C. for 3 hours, and then the resultant was concentrated under reduced pressure. Diethyl ether was added to the residue and the resultant was concentrated under reduced pressure to obtain a crude title compound (76.9 g).

ESI-MS m/z 162 [M+H]+

### (3) Synthesis of (R)-2-methyl-1,4-oxazepan-5-one

DBU (132 mL, 884 mmol) was added to a solution of the compound obtained in Production Example 2-(2) (76.9 g) in methanol (693 mL) at room temperature, and the mixture was heated under reflux for 16 hours. The reaction mixture was cooled to room temperature and then concentrated under reduced pressure. The resultant residue was purified by silica gel column chromatography (ethyl acetate/methanol) twice to obtain a title compound (21.5 g, 166 mmol).

¹H-NMR (400 MHz, CDCl₃) δ (ppm): 1.19 (d, J=6.3 Hz, 3H), 2.48-2.58 (m, 1H), 2.89 (ddd, J=2.5, 10.9, 15.4 Hz, 1H), 3.03 (ddd, J=0.9, 7.6, 15.3 Hz, 1H), 3.35 (ddd, J=3.9, 8.4, 15.4 Hz, 1H), 3.57-3.76 (m, 2H), 4.01 (ddd, J=2.5, 5.3, 12.7 Hz, 1H), 5.85-6.07 (m, 1H).

ESI-MS m/z 130 [M+H]+

### Production Example 3

### Synthesis of (S)-2-(fluoromethyl)-5-methoxy-2,3,6,7-tetrahydro-1,4-oxazepine

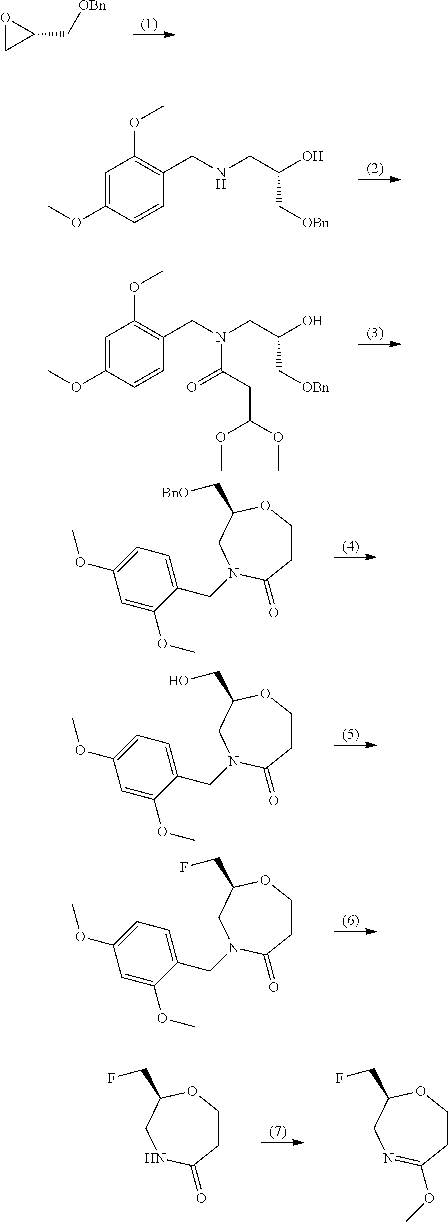

### (1) Synthesis of (S)-1-(benzyloxy)-3-((2,4-dimethoxybenzyl)amino)propan-2-ol

Lithium bis(trifluoromethanesulfonyl)imide (87 g, 304.5 mmol) was added to a solution of 2,4-dimethoxybenzylamine (CAS No. 20781-20-8; 46.7 mL, 310.6 mmol) and (S)-(+)-benzyl glycidyl ether (CAS No. 16495-13-9; 50.0 g, 304.5 mmol) in DCM (1.0 L) under water-cooling. The reaction mixture was stirred at room temperature for 20 hours. Water was added to the reaction mixture to separate the organic layer. The organic layers were dried over anhydrous magnesium sulfate. The solvent was evaporated under reduced pressure to obtain a crude title compound (119.4 g).

ESI-MS m/z 332 [M+H]+

### (2) Synthesis of (S)—N-(3-(benzyloxy)-2-hydroxypropyl)-N-(2,4-dimethoxybenzyl)-3,3-dimethoxypropanamide

EDC (88 g, 456.7 mmol) and HOBT (456.7 mmol) were added to a solution of the compound obtained in Production Example 3-(1) (119.4 g), 3,3-dimethoxypropionic acid (47.0 g, 350.1 mmol), and DIPEA (159 mL) in DMF (800 mL) at room temperature. The reaction mixture was stirred for 16 hours, and then ethyl acetate and a saturated aqueous sodium chloride solution were added. The organic layer was separated and washed with a saturated aqueous sodium chloride solution. The organic layer was dried over anhydrous magnesium sulfate. The organic layer was filtered through a silica gel pad (NH silica gel+silica gel, ethyl acetate). The resultant filtrate was concentrated under reduced pressure to obtain a crude title compound (125.5 g).

ESI-MS m/z 470 [M+Na]+

### (3) Synthesis of (S)-2-((benzyloxy)methyl)-4-(2,4-dimethoxybenzyl)-3,4-dihydro-1,4-oxazepin-5(2H)-one

A solution of the compound obtained in Production Example 3-(2) (125.5 g) and PPTS (35.2 g, 140.2 mmol) in xylene (1 L) was heated under reflux for 6 hours. The reaction mixture was cooled to room temperature, and ethyl acetate and a saturated aqueous sodium bicarbonate solution were added to the reaction mixture to separate the organic layer. The organic layer was washed with a saturated aqueous sodium chloride solution and then dried over anhydrous magnesium sulfate. The organic layer was concentrated under reduced pressure and the resultant residue was purified by column chromatography (n-heptane/ethyl acetate) to obtain a title compound (57.7 g, 150 mmol).

ESI-MS m/z 384 [M+H]+, 406 [M+Na]+

### (4) Synthesis of (S)-4-(2,4-dimethoxybenzyl)-2-(hydroxymethyl)-1,4-oxazepan-5-one

A mixture of the compound obtained in Production Example 3-(3) (57.7 g, 150.5 mmol), 20% palladium hydroxide/carbon (6 g, including 50% water content), acetic acid (20 mL), and ethanol (600 mL) was stirred under hydrogen atmosphere at 4 to 5 MPa and 70° C. for 50 hours. The reaction mixture was cooled to room temperature. The insolubles were filtered off through Celite (trademark) and the resultant was washed with ethyl acetate. The filtrate was concentrated under reduced pressure. The resultant residue was purified by silica gel column chromatography (n-heptane/ethyl acetate→ethyl acetate/methanol) to obtain a title compound (33.7 g).

¹H-NMR (400 MHz, CDCl₃) δ (ppm): 1.83 (dd, J=5.1, 7.0 Hz, 1H), 2.63 (dd, J=5.1, 15.2 Hz, 1H), 2.95 (ddd, J=2.7, 11.3, 15.6 Hz, 1H), 3.22-3.30 (m, 2H), 3.40-3.45 (m, 2H), 3.51 (dd, J=8.2, 16.0 Hz, 1H), 3.62-3.67 (m, 1H), 3.80 (s, 3H), 3.81 (s, 3H), 4.04 (ddd, J=2.3, 5.1, 12.5 Hz, 1H), 4.36 (d, J=14.5 Hz, 1H), 4.73 (d, J=14.5 Hz, 1H), 6.43-6.47 (m, 2H), 7.22 (d, J=8.6 Hz, 1H).

ESI-MS m/z 296 [M+H]+, 318 [M+Na]+

### (5) Synthesis of (S)-4-(2,4-dimethoxybenzyl)-2-(fluoromethyl)-1,4-oxazepan-5-one

Perfluorobutanesulfonyl fluoride (45.1 mL, 251.0 mmol) was added to a solution of the compound obtained in Production Example 3-(4) (33.7 g, 114.1 mmol), DIPEA (49.2 mL, 285.3 mmol), and tetrabutylammonium difluorotriphenyl silicate (73.9 g, 136.9 mmol) in THF (600 mL) at room temperature. The reaction mixture was stirred at room temperature for 64 hours. The reaction mixture was concentrated under reduced pressure. A mixed solvent of toluene/ethyl acetate (5/1) and a saturated aqueous sodium chloride solution were added to the resultant residue to separate the organic layer. The organic layer was further washed with a saturated aqueous sodium chloride solution twice. The organic layer was concentrated under reduced pressure and the resultant residue was purified serially by silica gel column chromatography (n-heptane/ethyl acetate) and NH silica gel column chromatography (n-heptane/ethyl acetate) to obtain a crude title compound (41 g).

¹H-NMR (400 MHz, CDCl₃) δ (ppm): 2.62 (dd, J=5.5, 15.2 Hz, 1H), 2.96 (ddd, J=2.3, 11.3, 15.2 Hz, 1H), 3.35-3.68 (m, 4H), 3.80 (s, 3H), 3.81 (s, 3H), 4.00 (ddd, J=2.3, 5.1, 12.5 Hz, 1H), 4.09-4.36 (m, 2H), 4.40 (d, J=14.5 Hz, 1H), 4.74 (d, J=14.5 Hz, 1H), 6.44-6.47 (m, 2H), 7.24 (d, J=8.2 Hz, 1H).

ESI-MS m/z 298 [M+H]+

### (6) Synthesis of (S)-2-(fluoromethyl)-1,4-oxazepan-5-one

Triethylsilane (27.4 mL, 171.7 mmol) was added to a solution of the compound obtained in Production Example 3-(5) (41 g) in TFA (300 mL) at room temperature. The reaction mixture was stirred at 60° C. for 3 hours. The reaction mixture was concentrated under reduced pressure. The resultant residue was purified by silica gel column chromatography (n-heptane/ethyl acetate→ethyl acetate/methanol) to obtain a title compound (15 g, 101.94 mmol).

¹H-NMR (400 MHz, CDCl₃) δ (ppm): 2.54 (ddd, J=2.0, 5.1, 15.6 Hz, 1H), 2.93 (ddd, J=2.7, 11.3, 15.6 Hz, 1H), 3.23-3.31 (m, 1H), 3.46 (ddd, J=3.5, 8.6, 15.2 Hz, 1H), 3.66-3.78 (m, 2H), 4.07 (ddd, J=2.7, 5.1, 12.5 Hz, 1H), 4.24-4.53 (m, 2H), 6.50 (brs, 1H).

### (7) Synthesis of (S)-2-(fluoromethyl)-5-methoxy-2,3,6,7-tetrahydro-1,4-oxazepine

Trimethyloxonium tetrafluoroborate (17.34 g, 117.2 mmol) was added to a solution of the compound obtained in Production Example 3-(6) (15 g, 101.94 mmol) in DCM (400 mL) at room temperature. The reaction solution was stirred at room temperature for 14 hours. A saturated aqueous sodium bicarbonate solution was added to the reaction mixture, and the mixture was stirred at room temperature for 30 minutes. Chloroform was added to the mixture to separate the organic layer. The organic layer was dried over anhydrous magnesium sulfate. The organic layer was concentrated under reduced pressure to obtain a title compound (14.9 g, 93 mmol).

¹H-NMR (400 MHz, CDCl₃) δ (ppm): 2.47 (ddd, J=1.2, 4.3, 15.6 Hz, 1H), 2.87-2.96 (m, 1H), 3.45-3.70 (m, 4H), 3.63 (s, 3H), 3.98 (ddd, J=3.1, 4.3, 12.1 Hz, 1H), 4.30-4.50 (m, 2H).

### Production Example 4

### Synthesis of (S)-2-(fluoromethyl)-1,4-oxazepan-5-one

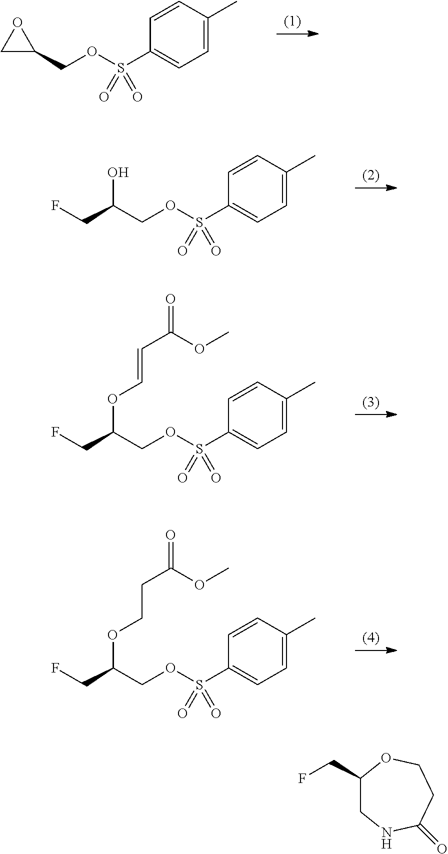 [SMILES: Cc1ccc(S(=O)(=O)OC[C@H]2CO2)cc1 ; Cc1ccc(S(=O)(=O)OC[C@H](O)CF)cc1 ; COC(=O)/C=C/O[C@H](CF)COS(=O)(=O)c1ccc(C)cc1 ; COC(=O)CCO[C@H](CF)COS(=O)(=O)c1ccc(C)cc1 ; O=C1CCO[C@H](CF)CN1]

### (1) Synthesis of (S)-3-fluoro-2-hydroxypropyl-4-methylbenzene sulfonate

Diethyl ether (1 L), (2R)-(−)-glycidyl tosylate (CAS No. 113826-06-5; 50.0 g, 219 mmol), and benzoyl fluoride (33.4 mL, 307 mmol) were added to a mixture of (R,R)-(−)-N,N′-bis(3,5-di-tert-butylsalicylidene)-1,2-cyclohexanediaminocobalt(II) (9.26 g, 15.3 mmol), HFIP (64.4 mL, 613 mmol), and DBN (1.51 mL, 12.3 mmol). The reaction mixture was stirred at room temperature overnight and then a 7 M ammonia/methanol solution (150 mL) was added. The mixture was stirred at room temperature for 2 hours and the solvent was evaporated under reduced pressure. Ethyl acetate (300 mL) was added to the resultant residue, and the resultant was washed serially with water and a saturated aqueous sodium chloride solution. The organic layer was dried over anhydrous magnesium sulfate, and the solvent was evaporated under reduced pressure. The resultant residue was purified by silica gel column chromatography (n-heptane/ethyl acetate) to obtain a title compound (45.5 g, 183 mmol).

¹H-NMR (400 MHz, CDCl₃) δ (ppm): 2.28-2.42 (m, 1H), 2.46 (s, 3H), 4.03-4.18 (m, 3H), 4.34-4.54 (m, 2H), 7.37 (d, J=8.2 Hz, 2H), 7.81 (d, J=8.2 Hz, 2H).

ESI-MS m/z 271 [M+Na]+

### (2) Synthesis of (S,E)-methyl 3-((1-fluoro-3-(tosyloxy)propan-2-yl)oxy)acrylate

A solution of the compound obtained in Production Example 4-(1) (45.5 g, 183 mmol), NMM (12.1 mL, 110 mmol), and methyl propionate (CAS No. 922-67-8; 19.8 mL, 238 mmol) in THF (315 mL) was stirred under ice-cooling for 3 hours. Acetic acid (6.29 mL, 110 mmol) was added to the reaction mixture, and then water and ethyl acetate were added. The organic layer was separated and washed serially with water and a saturated aqueous sodium chloride solution. The organic layer was dried over anhydrous magnesium sulfate and then concentrated under reduced pressure. The resultant residue was purified by silica gel column chromatography (n-heptane/ethyl acetate) to obtain a title compound (49.2 g, 148 mmol).

¹H-NMR (400 MHz, CDCl₃) δ (ppm): 2.46 (s, 3H), 3.70 (s, 3H), 4.11-4.37 (m, 3H), 4.42-4.66 (m, 2H), 5.26 (d, J=12.5 Hz, 1H), 7.33-7.42 (m, 3H), 7.76-7.83 (m, 2H).

ESI-MS m/z 355 [M+Na]+

### (3) Synthesis of (S)-methyl 3-((1-fluoro-3-(tosyloxy)propan-2-yl)oxy)propanoate

A suspension of the compound obtained in Production Example 4-(2) (48.8 g, 147 mmol) and 5% palladium/carbon (6.25 g, including 50% water content) in ethanol (279 mL) was stirred under hydrogen atmosphere at room temperature for 2 hours. The insolubles were removed, and then the filtrate was concentrated under reduced pressure to obtain a crude title compound (45.8 g).

¹H-NMR (400 MHz, CDCl₃) δ (ppm): 2.46 (s, 3H), 2.53 (t, J=6.3 Hz, 2H), 3.68 (s, 3H), 3.27-3.87 (m, 3H), 4.08 (dt, J=1.6, 5.5 Hz, 2H), 4.29-4.53 (m, 2H), 7.36 (d, J=8.2 Hz, 2H), 7.80 (d, J=8.2 Hz, 2H).

ESI-MS m/z 357 [M+Na]+

### (4) Synthesis of (S)-2-(fluoromethyl)-1,4-oxazepan-5-one

A mixture of the compound obtained in Production Example 4-(3) (45.8 g, 137 mmol) and a 7 M ammonia/methanol solution (391 mL, 2.74 mol) was stirred in an autoclave at 130° C. for 2 hours. The reaction mixture was cooled to room temperature, and then the mixture was concentrated under reduced pressure. Methanol (300 mL) and DBU (41.0 mL, 274 mmol) were added to the residue at room temperature. The reaction mixture was stirred at 100° C. for 3 hours. The reaction mixture was cooled to room temperature and then concentrated under reduced pressure. The resultant residue was purified by silica gel column chromatography (n-heptane/ethyl acetate→ethyl acetate/methanol) to obtain a title compound (10.4 g, 70.7 mmol).

¹H-NMR (400 MHz, CDCl₃) δ (ppm): 2.54 (ddd, J=2.0, 5.1, 15.6 Hz, 1H), 2.93 (ddd, J=2.7, 11.3, 15.6 Hz, 1H), 3.23-3.31 (m, 1H), 3.46 (ddd, J=3.5, 8.6, 15.2 Hz, 1H), 3.66-3.78 (m, 2H), 4.07 (ddd, J=2.7, 5.1, 12.5 Hz, 1H), 4.24-4.53 (m, 2H), 6.50 (brs, 1H).

### Production Example 5

### Synthesis of (R)-methyl 3-chloro-6-methyl-5,6,8,9-tetrahydroimidazo[1,5-d][1,4]oxazepine-1-carboxylate

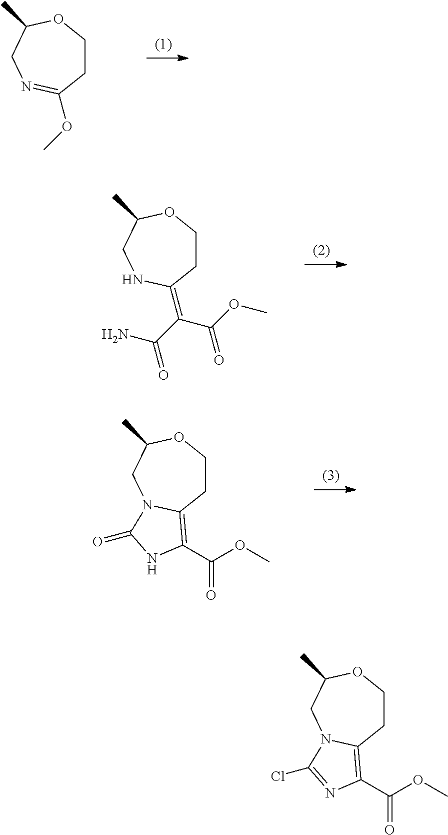

### (1) Synthesis of (R)-methyl 3-amino-2-(2-methyl-1,4-oxazepan-5-ylidene)-3-oxopropanoate

A solution of the compound obtained in Production Example 1-(6) (16.0 g, 156 mmol) and methyl carbamoyl acetate (CAS No. 51513-29-2; 18.3 g, 156 mmol) in THF (40 mL)/DMF (10 mL) was stirred at 90° C. for 15 hours. The reaction mixture was cooled to room temperature and the solvent was evaporated under reduced pressure. The resultant residue was purified by silica gel chromatography (n-heptane/ethyl acetate→ethyl acetate/methanol) to obtain a title compound (14.2 g, 62.2 mmol).

¹H-NMR (400 MHz, CDCl₃) δ (ppm): 1.20 (d, J=6.3 Hz, 3H), 2.73-2.81 (m, 1H), 3.33-3.66 (m, 5H), 3.77 (s, 3H), 4.04-4.10 (m, 1H).

### (2) Synthesis of (R)-methyl 6-methyl-3-oxo-2,3,5,6,8,9-hexahydroimidazo[1,5-d][1,4]oxazepine-1-carboxylate

Iodobenzene diacetate (24.1 g, 74.7 mmol) was added to a solution of the compound obtained in Production Example 5-(1) (14.2 g, 62.2 mmol) in THF (100 mL)/toluene (100 mL), and the mixture was stirred at room temperature for 60 hours. A saturated aqueous sodium bicarbonate solution (60 mL) and a saturated aqueous sodium sulfite solution (60 mL) were added to the reaction mixture, and the mixture was stirred at room temperature for 1 hour. The mixture was extracted with ethyl acetate three times. The combined organic layers were dried over anhydrous magnesium sulfate and concentrated under reduced pressure. The resultant residue was purified by silica gel column chromatography (n-heptane/ethyl acetate→ethyl acetate/methanol) to obtain a title compound (9.97 g, 44.1 mmol).

¹H-NMR (400 MHz, CDCl₃) δ (ppm): 1.27 (d, J=6.3 Hz, 3H), 2.86 (ddd, J=2.4, 11.0, 16.3 Hz, 1H), 3.45 (dd, J=9.0, 14.7 Hz, 1H), 3.53-3.70 (m, 3H), 3.83 (s, 3H), 4.13-4.19 (m, 1H), 4.29 (d, J=14.7 Hz, 1H), 8.03 (brs, 1H).

ESI-MS m/z 227 [M+H]+

### (3) Synthesis of (R)-methyl 3-chloro-6-methyl-5,6,8,9-tetrahydroimidazo[1,5-d][1,4]oxazepine-1-carboxylate

A mixture of the compound obtained in Production Example 5-(2) (9.97 g, 44.1 mmol) and phosphorus oxychloride (60 mL) was stirred at 110° C. for 4 hours. The reaction mixture was cooled to room temperature and concentrated under reduced pressure. The resultant residue was purified by NH silica gel column chromatography (n-heptane/ethyl acetate) to obtain a title compound (5.94 g, 24.3 mmol).

¹H-NMR (400 MHz, CDCl₃) δ (ppm): 1.30 (d, J=6.5 Hz, 3H), 3.02 (ddd, J=2.7, 10.8, 16.4 Hz, 1H), 3.55-3.62 (m, 1H), 3.66-3.74 (m, 1H), 3.87 (s, 3H), 3.88-3.98 (m, 2H), 4.13-4.19 (m, 1H), 4.26-4.31 (m, 1H).

ESI-MS m/z 245 [M+H]+

### Production Example 6

### Synthesis of (S)-methyl 3-chloro-6-(fluoromethyl)-5,6,8,9-tetrahydroimidazo[1,5-d][1,4]oxazepine-1-carboxylate

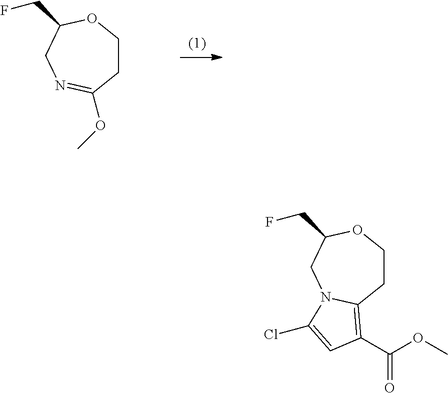 [SMILES: COC1=NC[C@@H](CF)OCC1 ; COC(=O)c1cc(Cl)n2c1CCO[C@H](CF)C2]

According to Production Examples 8-(1), 8-(2), and 8-(3), a title compound (1.77 g, 6.74 mmol) was obtained from the compound obtained in Production Example 3-(7) (9.39 g, 58.3 mmol).

¹H-NMR (400 MHz, CDCl₃) δ (ppm): 3.02 (ddd, J=2.7, 11.4, 16.4 Hz, 1H), 3.58-3.65 (m, 1H), 3.71-3.80 (m, 1H), 3.88 (s, 3H), 3.98-4.09 (m, 2H), 4.23-4.28 (m, 1H), 4.33-4.65 (m, 3H).

ESI-MS m/z 263 [M+H]+

### Production Example 7

### Synthesis of N-cyclobutyl-4-(4,4,5,5-tetramethyl-1,3,2-dioxaborolan-2-yl)benzenesulfonamide

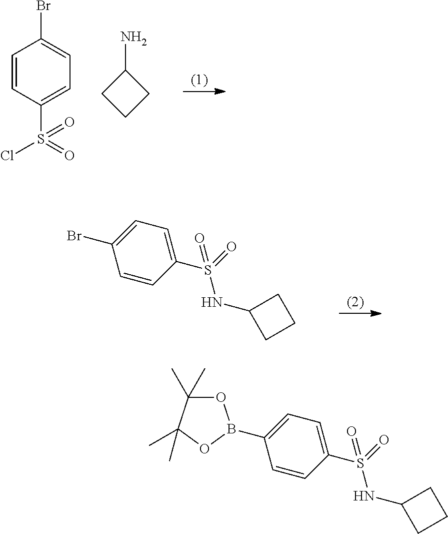

### (1) Synthesis of 4-bromo-N-cyclobutylbenzenesulfonamide

To a DCM solution (10 mL) of cyclobutylamine (CAS No. 2516-34-9; 306 mg, 4.31 mmol) and TEA (0.818 mL, 5.87 mmol), 4-bromobenzenesulfonyl chloride (CAS No. 98-58-8; 1 g, 3.91 mmol) was added under ice cooling. The reaction solution was returned to room temperature and stirred for 20 hours. Water was added to the reaction solution and the organic layer was separated. The organic layer was dried over anhydrous magnesium sulfate, and the solvent was distilled away under reduced pressure. The residue was purified by silica gel column chromatography (n-heptane/ethyl acetate) to obtain the title compound (742 mg, 2.56 mmol).

¹H-NMR (400 MHz, CDCl₃) δ (ppm): 1.56-1.69 (m, 2H), 1.70-1.88 (m, 2H), 2.09-2.28 (m, 2H), 3.69-3.91 (m, 1H), 4.60 (d, J=9.0 Hz, 1H), 7.60-7.67 (m, 2H), 7.69-7.80 (m, 2H).

### (2) Synthesis of N-cyclobutyl-4-(4,4,5,5-tetramethyl-1,3,2-dioxaborolan-2-yl)benzenesulfonamide

To a DMF solution (20 mL) of the compound obtained in production example 7-(1) (740 mg, 2.55 mmol), potassium acetate (1.00 g, 10.2 mmol), and bis(pinacolato)diboron (1.94 g, 7.65 mmol), Pd(dppf)Cl₂.CH₂Cl₂ (93 mg, 128 μmol) was added at room temperature. The reaction mixture was stirred at 90° C. for 3 hours, then cooled to room temperature, and the insoluble matter was filtered off. The filtrate was diluted with ethyl acetate, then washed with water once, and subsequently washed with a saturated aqueous sodium chloride solution twice. The organic layer was dried over anhydrous magnesium sulfate, and the solvent was distilled away under reduced pressure. The obtained residue was purified by silica gel column chromatography (n-heptane/ethyl acetate) to obtain the title compound (850 mg, 2.52 mmol).

¹H-NMR (400 MHz, CDCl₃) δ (ppm): 1.36 (s, 12H), 1.46-1.65 (m, 2H), 1.66-1.82 (m, 2H), 2.07-2.17 (m, 2H), 3.76-3.86 (m, 1H), 4.58 (d, J=9.0 Hz, 1H), 7.73-7.87 (m, 2H), 7.89-7.98 (m, 2H).

### Example 1

### Synthesis of (R)-4-(3-(4-fluorophenyl)-6-methyl-5,6,8,9-tetrahydroimidazo[1,5-d][1,4]oxazepin-1-yl)-N-isopropylbenzenesulfonamide

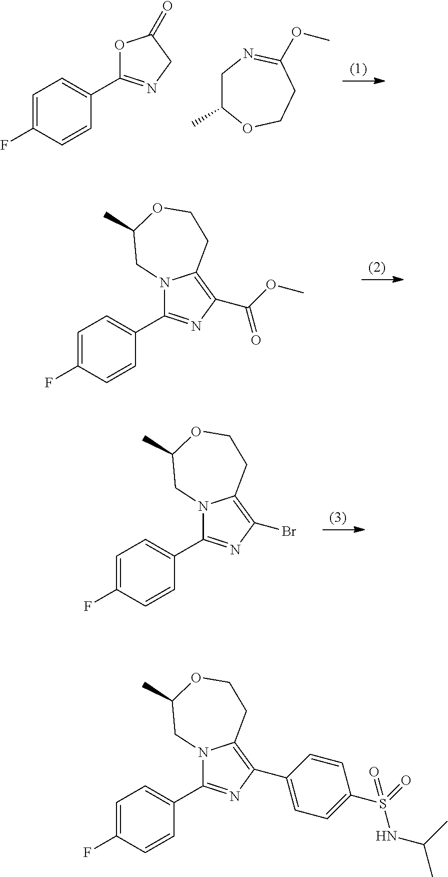

### (1) Synthesis of (R)-methyl 3-(4-fluorophenyl)-6-methyl-5,6,8,9-tetrahydroimidazo[1,5-d][1,4]oxazepine-1-carboxylate

A toluene solution (50 mL) of the compound obtained in production example 1-(6) (1.0 g, 6.98 mmol) and 2-(4-fluorophenyl)oxazol-5(4H)-one (CAS No. 105669-21-4; 1.38 g, 7.68 mmol) was heated under reflux for 3 hours. The reaction solution was cooled to room temperature, and the solvent was evaporated under reduced pressure. The obtained residue was dissolved in methanol (50 mL), to which sodium methoxide (490 mg, 9.08 mmol) was then added and the mixture was heated under reflux for 3 hours. The reaction solution was cooled to room temperature and partitioned between ethyl acetate and an aqueous ammonium chloride solution under ice cooling. The organic layer was washed with a saturated aqueous sodium chloride solution and dried over anhydrous magnesium sulfate, and the solvent was evaporated under reduced pressure. The residue was purified by NH silica gel column chromatography (n-heptane/ethyl acetate) to obtain a crude purified product (875 mg) including the title compound.

ESI-MS m/z 305[M+H]⁺

### (2) Synthesis of (R)-1-bromo-3-(4-fluorophenyl)-6-methyl-5,6,8,9-tetrahydroimidazo[1,5-d][1,4]oxazepine

An ethanol solution (15 mL) of the compound obtained in Example 1-(1) (875 mg) and a 5 N sodium hydroxide (1.15 mL, 5.75 mmol) was stirred at 60° C. for 1 hour. The reaction solution was acidified with a 5 N hydrochloric acid, and the solvent was evaporated under reduced pressure. Ethanol was added to the residue, the insoluble material was filtered off, and the filtrate was concentrated under reduced pressure. The obtained residue was dissolved in DMF (15 mL) and ethanol (5 mL), to which potassium carbonate (993 mg, 7.19 mmol) and NBS (768 mg, 4.31 mmol) were then added and the mixture was stirred at room temperature for 15 hours. The reaction mixture was diluted with ethyl acetate and a saturated aqueous sodium chloride solution. The organic layer was washed with a saturated aqueous sodium chloride solution and dried over anhydrous magnesium sulfate. The solvent was evaporated under reduced pressure, and the residue was purified by NH silica gel column chromatography (n-heptane/ethyl acetate) to obtain the title compound (310 mg, 0.953 mmol).

¹H-NMR (500 MHz, CDCl₃) δ (ppm): 1.22 (d, J=6.3 Hz, 3H), 2.95-3.14 (m, 2H), 3.61 (td, J=11.5, 1.5 Hz, 1H), 3.67-3.75 (m, 1H), 3.93 (dd, J=14.9, 8.5 Hz, 1H), 4.18 (d, J=14.6 Hz, 2H), 7.09-7.19 (m, 2H), 7.41-7.51 (m, 2H).

ESI-MS m/z 325[M+H]⁺, 327[M+H]⁺

### (3) Synthesis of (R)-4-(3-(4-fluorophenyl)-6-methyl-5,6,8,9-tetrahydroimidazo[1,5-d][1,4]oxazepin-1-yl)-N-isopropylbenzenesulfonamide

A DME solution (7 mL) of the compound obtained in Example 1-(2) (165 mg, 0.507 mmol), 4-(N-isopropylsulfamoyl)phenylboronic acid (173 mg, 0.710 mmol), (A-taPhos)₂PdCl₂ (35.9 mg, 0.051 mmol), and a 1 N aqueous sodium carbonate solution (2 mL) was stirred under microwave irradiation at 140° C. for 60 minutes. The reaction mixture was purified by NH silica gel column chromatography (n-heptane/ethyl acetate to ethyl acetate/methanol) to obtain the title compound (160 mg, 0.361 mmol).

¹H-NMR (500 MHz, CDCl₃) δ (ppm): 1.09 (d, J=6.3 Hz, 6H), 1.26 (d, J=6.3 Hz, 3H), 3.15-3.24 (m, 1H), 3.29-3.35 (m, 1H), 3.48 (dq, J=13.7, 6.6 Hz, 1H), 3.68 (t, J=11.2 Hz, 1H), 3.75-3.83 (m, 1H), 3.99 (dd, J=14.6, 8.3 Hz, 1H), 4.18-4.28 (m, 3H), 7.18 (t, J=8.8 Hz, 2H), 7.51 (dd, J=8.5, 5.1 Hz, 2H), 7.74 (d, J=8.8 Hz, 2H), 7.89 (d, J=8.8 Hz, 2H).

ESI-MS m/z 444[M+H]⁺

### Example 2

### Synthesis of (S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide

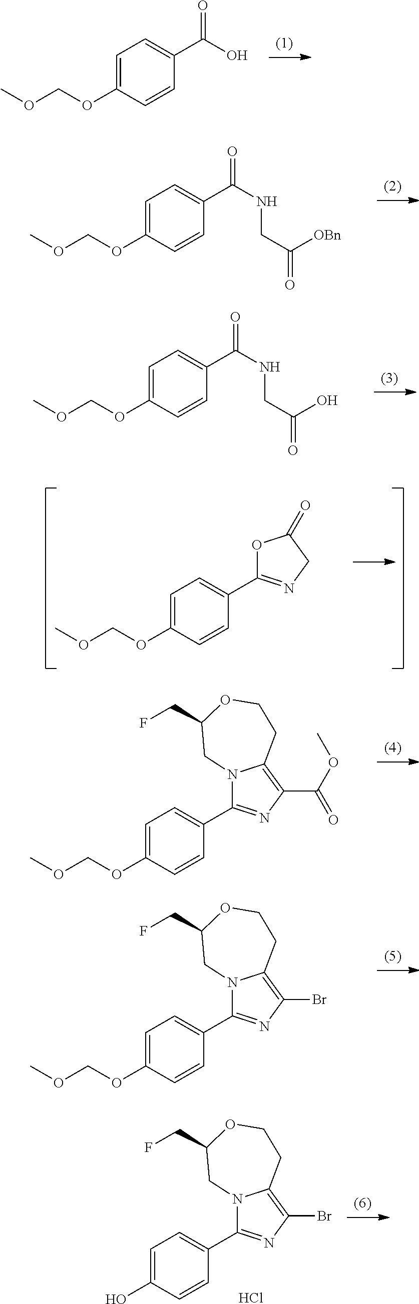

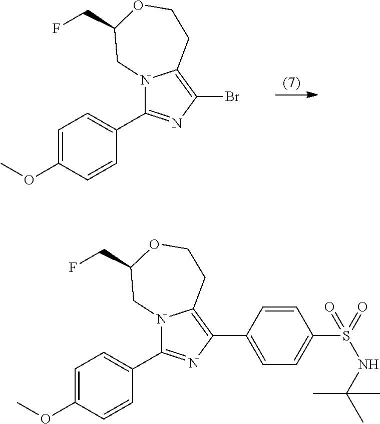 [SMILES: COc1ccc(-c2nc(Br)c3n2C[C@@H](CF)OCC3)cc1 ; COc1ccc(-c2nc(-c3ccc(S(=O)(=O)NC(C)(C)C)cc3)c3n2C[C@@H](CF)OCC3)cc1]

### (1) Synthesis of benzyl 2-(4-(methoxymethoxy)benzamide)acetate

To a DCM solution (200 mL) of glycine benzyl ester p-toluenesulfonate (CAS No. 1738-76-7; 18.3 g, 54.3 mmol), 4-methoxymethoxy benzoic acid (CAS No. 25458-44-0; 9.0 g, 49.4 mmol), and DIPEA (25.8 ml, 148 mmol), EDC (14.2 g, 74.1 mmol) and HOBT (10.0 g, 74.1 mmol) were added. The reaction solution was stirred at room temperature for 18 hours, and then partitioned between chloroform and a saturated aqueous sodium chloride solution. The organic layer was washed with a saturated aqueous sodium chloride solution and dried over anhydrous magnesium sulfate. The organic layer was concentrated and the residue was purified by silica gel column chromatography (n-heptane/ethyl acetate) to obtain the title compound (14.1 g, 42.8 mmol).

¹H-NMR (400 MHz, CDCl₃) δ (ppm): 3.48 (s, 3H), 4.28 (d, J=5.1 Hz, 2H), 5.22 (s, 2H), 5.23 (s, 2H), 6.55 (brs, 1H), 7.00-7.15 (m, 2H), 7.28-7.51 (m, 5H), 7.71-7.87 (m, 2H).

### (2) Synthesis of 2-(4-(methoxymethoxy)benzamide)acetic acid

An ethanol suspension (300 mL) of the compound obtained in Example 2-(1) (14.1 g, 42.8 mmol) and 10% palladium-carbon (product having a water content of 50 wt %; 1.5 g) was stirred under a hydrogen atmosphere at room temperature for 4 hours. The insoluble material was filtered off, and the filtrate was concentrated under reduced pressure to obtain the title compound (10.2 g, 42.8 mmol).

¹H-NMR (400 MHz, CD₃OD) δ (ppm): 3.45 (s, 3H), 4.07 (s, 2H), 5.24 (s, 2H), 6.98-7.25 (m, 2H), 7.61-8.01 (m, 2H).

### (3) Synthesis of (S)-methyl 6-(fluoromethyl)-3-(4-(methoxymethoxy)phenyl)-5,6,8,9-tetrahydroimidazo[1,5-d][1,4]oxazepine-1-carboxylate

To a THF solution (40 mL) of the compound obtained in Example 2-(2) (3.00 g, 12.5 mmol) and NMM (1.45 mL, 13.2 mmol), methyl chloroformate (1.02 mL, 13.2 mmol) was added dropwise at −10° C. The reaction mixture was stirred at the same temperature for 1 hour, and then stirred for 2 hours while slowly warmed to room temperature. The resulting amine hydrochloride was filtered off on celite, and the filtrate was concentrated under reduced pressure. The obtained residue and the compound obtained in production example 3-(7) (2.02 g, 12.5 mmol) were dissolved in toluene (50 mL), and the mixture was heated under reflux for 6 hours. The reaction solution was cooled to room temperature, and the solvent was evaporated under reduced pressure. The residue was dissolved in methanol (40 mL), to which sodium methoxide (677 mg, 12.5 mmol) was then added. The reaction solution was heated under reflux for 2.5 hours, then cooled to room temperature, and partitioned between ethyl acetate and an aqueous ammonium chloride solution, and the organic layer was washed with a saturated aqueous sodium chloride solution. The organic layer was dried over anhydrous magnesium sulfate, and the solvent was evaporated under reduced pressure. The residue was purified by silica gel column chromatography (n-heptane/ethyl acetate) to obtain a crude purified product (2.15 g) containing the title compound.

ESI-MS m/z 365[M+H]⁺

### (4) Synthesis of (S)-1-bromo-6-(fluoromethyl)-3-(4-(methoxymethoxy)phenyl)-5,6,8,9-tetrahydroimidazo[1,5-d][1,4]oxazepine

From the compound obtained in Example 2-(3) (2.15 g), the title compound (1.15 g, 2.99 mmol) was obtained according to the method in Example 1-(2).

¹H-NMR (400 MHz, CDCl₃) δ (ppm): 2.91-3.16 (m, 2H), 3.49 (s, 3H), 3.59-3.86 (m, 2H), 3.99 (dd, J=14.6, 8.8 Hz, 1H), 4.19-4.72 (m, 4H), 5.21 (s, 2H), 7.10 (d, J=9.0 Hz, 2H), 7.40 (d, J=9.0 Hz, 2H).

ESI-MS m/z 385[M+H]⁺, 387[M+H]⁺

### (5) Synthesis of (S)-4-(1-bromo-6-(fluoromethyl)-5,6,8,9-tetrahydroimidazo[1,5-d][1,4]oxazepin-3-yl)phenol hydrochloride

A methanol solution (15 mL) of the compound obtained in Example 2-(4) (1.15 g, 2.99 mmol) and a concentrated hydrochloric acid (1 mL, 12 mmol) was stirred at 60° C. for 3 hours. The reaction solution was cooled to room temperature, and the solvent was evaporated under reduced pressure to obtain the title compound (1.09 g, 2.89 mmol).

ESI-MS m/z 341[M+H]⁺, 343 [M+H]⁺

### (6) Synthesis of (S)-1-bromo-6-(fluoromethyl)-3-(4-methoxyphenyl)-5,6,8,9-tetrahydroimidazo[1,5-d][1,4]oxazepine

A mixture of the compound obtained in Example 2-(5) (100 mg, 0.265 mmol), cesium carbonate (259 mg, 0.794 mmol), methyl p-toluenesulfonate (74 mg, 0.397 mmol), and DMF (4 mL) was stirred at 80° C. for 3 hours. The reaction solution was cooled to room temperature, to which ethyl acetate and water were then added. The organic layer was separated, washed with a saturated aqueous sodium chloride solution, and dried over anhydrous magnesium sulfate, and the solvent was evaporated under reduced pressure. The obtained residue was purified by NH silica gel column chromatography (n-heptane/ethyl acetate) to obtain the title compound (82 mg, 0.231 mmol).

ESI-MS m/z 355[M+H], 357[M+H]⁺

### (7) Synthesis of (S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide

From the compound obtained in Example 2-(6) (27 mg, 0.076 mmol) and 4-(tert-butylaminosulfonyl)benzeneboronic acid (29.3 mg, 0.114 mmol), the title compound (22 mg, 0.045 mmol) was obtained according to the method in Example 1-(3).

¹H-NMR (400 MHz, CDCl₃) δ (ppm): 1.23 (s, 9H), 3.21 (ddd, J=16.3, 11.0, 2.3 Hz, 1H), 3.30-3.41 (m, 1H), 3.70 (t, J=11.3 Hz, 1H), 3.81-3.92 (m, 1H), 3.86 (s, 3H), 4.04 (dd, J=14.6, 8.8 Hz, 1H), 4.23-4.63 (m, 5H), 6.96-7.04 (m, 2H), 7.42-7.50 (m, 2H), 7.70-7.77 (m, 2H), 7.86-7.93 (m, 2H).

ESI-MS m/z 488[M+H]⁺

### Example 3

### Synthesis of (R)—N-cyclobutyl-4-(3-(4-methoxyphenyl)-6-methyl-5,6,8,9-tetrahydroimidazo[1,5-d][1,4]oxazepin-1-yl)benzenesulfonamide

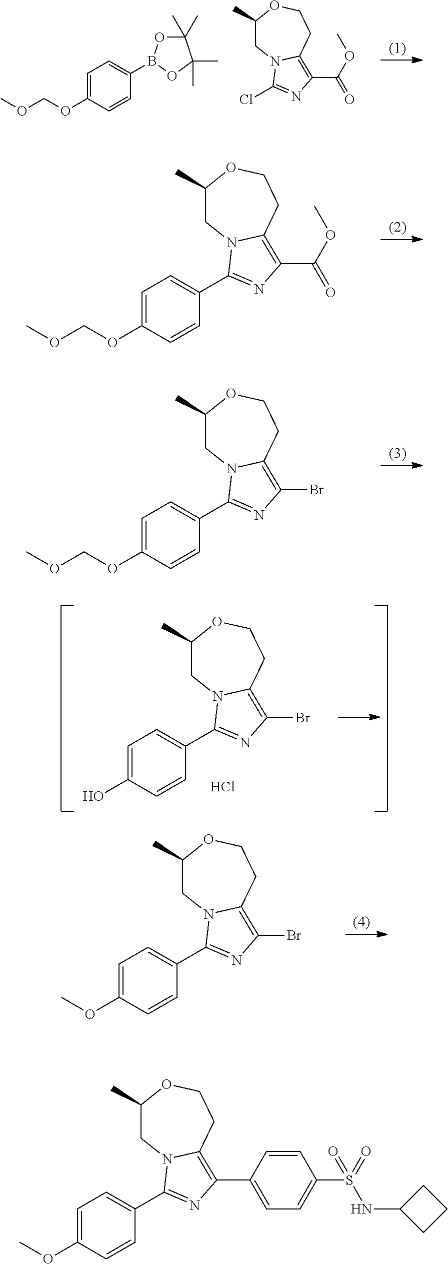

### (1) Synthesis of (R)-methyl 3-(4-(methoxymethoxy)phenyl)-6-methyl-5,6,8,9-tetrahydroimidazo[1,5-d][1,4]oxazepine-1-carboxylate

A mixture of the compound obtained in production example 5-(3) (1 g, 4.09 mmol), 2-(4-(methoxymethoxy)phenyl)-4,4,5,5-tetramethyl-1,3,2-dioxaborolane (CAS No. 936250-15-6; 2.70 g, 10.2 mmol), tetrakis(triphenylphosphine)palladium(0) (472 mg, 0.409 mmol), a 1 M aqueous sodium carbonate solution (7.36 mL), and DME (16 mL) was stirred under microwave irradiation at 130° C. for 30 minutes. Because no reaction proceeded, the reaction mixture was partitioned between ethyl acetate and a saturated aqueous sodium chloride solution to separate the organic layer. The aqueous layer was extracted with ethyl acetate three times. The combined organic layers were concentrated, and the residue was purified by silica gel column chromatography (n-heptane/ethyl acetate) to collect the raw material. A mixture of the collected raw material, 2-(4-(methoxymethoxy)phenyl)-4,4,5,5-tetramethyl-1,3,2-dioxaborolane (2.70 g, 10.2 mmol), (A-taPhos)₂PdCl₂ (232 mg, 0.327 mmol), a 1 M aqueous sodium carbonate solution (7.36 mL), and DME (16 mL) was stirred under microwave irradiation at 130° C. for 30 minutes. The reaction mixture was partitioned between ethyl acetate and a saturated aqueous sodium chloride solution to separate the organic layer. The aqueous layer was extracted with ethyl acetate three times. The combined organic layers were concentrated and the residue was purified by silica gel column chromatography (n-heptane/ethyl acetate) to obtain the title compound (1.07 g, 3.09 mmol).

¹H-NMR (400 MHz, CDCl₃) δ (ppm): 1.22 (d, J=6.6 Hz, 3H), 3.10 (ddd, J=16.4, 10.9, 2.3 Hz, 1H), 3.50 (s, 3H), 3.59-3.75 (m, 2H), 3.86-3.99 (m, 4H), 4.01-4.11 (m, 1H), 4.16-4.26 (m, 2H), 5.22 (s, 2H), 7.11 (d, J=8.2 Hz, 2H), 7.41 (d, J=8.6 Hz, 2H).

ESI-MS m/z 347[M+H]⁺

### (2) Synthesis of (R)-1-bromo-3-(4-(methoxymethoxy)phenyl)-6-methyl-5,6,8,9-tetrahydroimidazo[1,5-d][1,4]oxazepine

From the compound obtained in Example 3-(1) (1.07 g, 3.089 mmol), the title compound (833 mg, 2.268 mmol) was obtained according to the method in Example 2-(4).

¹H-NMR (400 MHz, CDCl₃) δ (ppm): 1.22 (d, J=6.3 Hz, 3H), 2.91-3.01 (m, 1H), 3.02-3.10 (m, 1H), 3.50 (s, 3H), 3.56-3.64 (m, 1H), 3.69 (quin, J=7.1 Hz, 1H), 3.90 (dd, J=14.8, 8.6 Hz, 1H), 4.13-4.28 (m, 2H), 5.21 (s, 2H), 7.10 (d, J=9.0 Hz, 2H), 7.34-7.42 (m, 2H).

ESI-MS m/z 367[M+H]⁺

### (3) Synthesis of (R)-1-bromo-3-(4-methoxyphenyl)-6-methyl-5,6,8,9-tetrahydroimidazo[1,5-d][1,4]oxazepine

From the compound obtained in Example 3-(2) (833 mg, 2.268 mmol), the corresponding phenol compound (659 mg) was obtained according to the method in Example 2-(5). The obtained compound (163 mg) was methylated according to the method in Example 2-(6) to obtain the title compound (130 mg, 0.386 mmol).

ESI-MS m/z 337[M+H]⁺

### (4) Synthesis of (R)—N-cyclobutyl-4-(3-(4-methoxyphenyl)-6-methyl-5,6,8,9-tetrahydroimidazo[1,5-d][1,4]oxazepin-1-yl)benzenesulfonamide

From the compound obtained in Example 3-(3) (21 mg, 0.062 mmol) and the compound obtained in production example 7-(2) (42 mg, 0.125 mmol), the title compound (14.1 mg, 0.030 mmol) was obtained according to the method in Example 1-(3).

¹H-NMR (400 MHz, CDCl₃) δ (ppm): 1.25 (d, J=6.6 Hz, 3H), 1.43-1.65 (m, 2H), 1.68-1.82 (m, 2H), 2.01-2.19 (m, 2H), 3.07-3.23 (m, 1H), 3.24-3.37 (m, 1H), 3.67 (dd, J=11.9, 10.4 Hz, 1H), 3.73-3.90 (m, 2H), 3.87 (s, 3H), 3.96 (dd, J=14.8, 8.2 Hz, 1H), 4.12-4.32 (m, 2H), 4.89 (d, J=9.0 Hz, 1H), 7.01 (d, J=9.0 Hz, 2H), 7.46 (d, J=8.6 Hz, 2H) 7.68-7.78 (m, 2H), 7.79-7.91 (m, 2H).

ESI-MS m/z 468[M+H]⁺

Each compound illustrated in Tables 1 and 2 was synthesized according to the method(s) of any of Examples described above.

TABLE 1

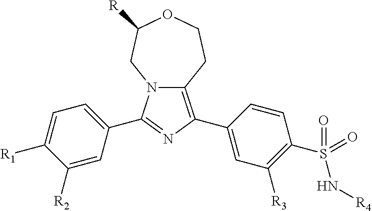

| Example | | | | | | ESI-MS |
|---|---|---|---|---|---|---|
| No. | R | R₁ | R₂ | R₃ | R₄ | [M + H]⁺ |
| 4 | CH₃ | OCH₂F | H | H | CH(CH₃)₂ | 474 |
| 5 | CH₃ | OCH₂F | H | H | tert-butyl | 488 |
| 6 | CH₃ | OCH₂CH₃ | H | H | CH(CH₃)₂ | 470 |
| 7 | CH₃ | OCH₂CH₃ | H | H | tert-butyl | 484 |
| 8 | CH₃ | OCH₃ | H | H | CH(CH₃)₂ | 456 |
| 9 | CH₃ | OCHF₂ | H | H | tert-butyl | 506 |
| 10 | CH₃ | OCH₃ | H | H | tert-butyl | 470 |
| 11 | CH₃ | 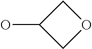 | H | H | CH(CH₃)₂ | 498 |
| 12 | CH₃ | OCHF₂ | H | H | CH(CH₃)₂ | 492 |
| 13 | CH₃ | F | H | H | tert-butyl | 458 |
| 14 | CH₃ | F | H | H | CH3 | 416 |
| 15 | CH₃ | F | H | H | CH2CH3 | 430 |
| 16 | CH₃ | F | H | H | cyclopropyl | 442 |
| 17 | CH₃ | F | H | H | 1-methyl- | 470 |
| | | | | | cyclobutyl | |
| 18 | CH₃ | OCH₂F | H | H | cyclopropyl | 472 |
| 19 | CH₃ | OCH₂F | H | H | cyclobutyl | 486 |
| 20 | CH₃ | OCH₂F | H | H | CH₃ | 446 |
| 21 | CH₃ | OCH₂F | H | H | CH₂CH₃ | 460 |
| 22 | CH₃ | OCH₂F | H | H | 1-methyl- | 500 |
| | | | | | cyclobutyl | |
| 23 | CH₃ | OCHF₂ | H | H | cyclopropyl | 490 |
| 24 | CH₃ | OCHF₂ | H | H | cyclobutyl | 504 |
| 25 | CH₃ | OCHF₂ | H | H | CH₃ | 464 |
| 26 | CH₃ | OCHF₂ | H | H | CH₂CH₃ | 478 |
| 27 | CH₃ | OCHF₂ | H | H | 1-methyl- | 518 |
| | | | | | cyclobutyl | |
| 28 | CH₃ | OCH₃ | H | H | cyclopropyl | 454 |
| 29 | CH₃ | OCH₃ | H | H | CH₃ | 428 |
| 30 | CH₃ | OCH₃ | H | H | CH₂CH₃ | 442 |
| 31 | CH₃ | OCH₃ | H | H | 1-methyl- | 482 |
| | | | | | cyclobutyl | |
| 32 | CH₃ | OCH₂CH₃ | H | H | cyclopropyl | 468 |
| 33 | CH₃ | OCH₂CH₃ | H | H | cyclobutyl | 482 |
| 34 | CH₃ | OCH₂CH₃ | H | H | CH₃ | 442 |
| 35 | CH₃ | OCH₂CH₃ | H | H | CH₂CH₃ | 456 |

TABLE 2

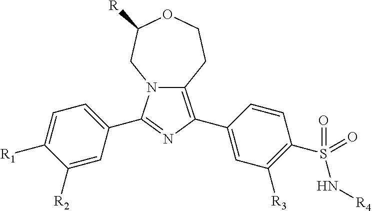

| Example | | | | | | ESI-MS |
|---|---|---|---|---|---|---|
| No. | R | R₁ | R₂ | R₃ | R₄ | [M + H]⁺ |
| 36 | CH₃ | OCH₂CH₃ | H | H | 1-methylcyclobutyl | 496 |
| 37 | CH₃ | F | H | H | cyclobutyl | 456 |
| 38 | CH₂F | F | H | H | cyclobutyl | 474 |
| 39 | CH₂F | F | H | H | 1-methylcyclobutyl | 488 |
| 40 | CH₂F | F | H | H | CH(CH₃)₂ | 462 |
| 41 | CH₂F | F | H | H | tert-butyl | 476 |
| 42 | CH₂F | F | H | H | CH₂CH₃ | 448 |
| 43 | CH₂F | OCH₃ | H | H | CH(CH₃)₂ | 474 |
| 44 | CH₂F | OCH₃ | H | H | cyclobutyl | 486 |
| 45 | CH₂F | OCH₃ | H | H | 1-methylcyclobutyl | 500 |
| 46 | CH₂F | OCH₃ | H | H | CH₂CH₃ | 460 |
| 47 | CH₂F | OCH₃ | H | H | cyclopropyl | 472 |
| 48 | CH₂F | OCH₂CH₃ | H | H | CH₂CH₃ | 488 |
| 49 | CH₂F | OCH₂CH₃ | H | H | cyclopropyl | 486 |
| 50 | CH₂F | OCH₂CH₃ | H | H | CH₂CH₃ | 474 |
| 51 | CH₂F | OCH₂F | H | H | CH(CH₃)₂ | 492 |
| 52 | CH₂F | OCH₂F | H | H | tert-butyl | 506 |
| 53 | CH₂F | OCHF₂ | H | H | CH(CH₃)₂ | 510 |
| 54 | CH₂F | OCHF₂ | H | H | tert-butyl | 524 |
| 55 | CH₂F | OCH₂CH₃ | H | H | tert-butyl | 502 |
| 56 | CH₂F | OCH₂F | H | H | CH₂CH₃ | 478 |
| 57 | CH₂F | OCH₂F | H | H | 1-methylcyclobutyl | 518 |
| 58 | CH₂F | OCH₂F | H | H | cyclobutyl | 504 |
| 59 | CH₂F | OCHF₂ | H | H | CH₂CH₃ | 496 |
| 60 | CH₂F | OCHF₂ | H | H | cyclobutyl | 522 |
| 61 | CH₂F | OCHF₂ | H | H | 1-methylcyclobutyl | 536 |
| 62 | CH₃ | OCH₃ | F | H | CH(CH₃)₂ | 474 |
| 63 | CH₃ | OCH₃ | F | H | tert-butyl | 488 |
| 64 | CH₃ | CH₃ | F | H | tert-butyl | 472 |
| 65 | CH₃ | CH₃ | F | H | CH(CH₃)₂ | 458 |
| 66 | CH₃ | OCHF₂ | F | H | tert-butyl | 524 |

### Test Example 1

### Affinity to mGluR2

(Preparation of Cell Membrane Fraction of HEK293 Cells Stably Expressing Human Metabotropic Glutamate Receptor 2 (mGluR2))

HEK293 cells stably expressing human mGluR2 and human glutamate transporter SLC1A3 were cultured in a Dulbecco's modified Eagle's medium with 10% fetal bovine serum (50 units/mL of penicillin, 50 μg/mL of streptomycin, 60 μg/mL of geneticin, 400 μg/mL of hygromycin B and 2 mM of glutamine) at 37° C. under 5% CO₂. Confluent cell cultures were washed twice with PBS(−), and then scraped off with a cell scraper, and subjected to centrifugal separation at 4° C. and 1500 rpm for 5 minutes for collecting cells. The centrifuged sediment (cell pellet) was homogenized in a 20 mM HEPES buffer containing 10 mM EDTA (pH 7.4) by using sonicator and centrifuged at 4° C. and 1500×g for 30 minutes. The supernatant (soluble fraction) was subjected to the centrifugal separation at 4° C. and 40,000×g for 30 minutes, and thus, insoluble fraction was obtained. After additional centrifugally washing the obtained fraction with 20 mM HEPES buffer containing 10 mM EDTA (pH 7.4), the pellet was centrifugally suspended with a 20 mM HEPES buffer containing 0.1 mM EDTA, and the cell membrane fraction was obtained by the centrifugal separation at 4° C. and 40,000×g for 30 minutes. The thus obtained cell membrane fraction was suspended in a 20 mM HEPES buffer containing 0.1 mM EDTA in a protein concentration of 3 mg/mL, which was stored at −80° C.

([³⁵S]GTPγS binding assay)

The frozen cell membrane fraction prepared as described above was thawed before use, and the resultant was diluted with a buffer for a binding assay (final concentrations: 20 mM HEPES, 100 mM NaCl, 1 mM MgCl₂, 3 μM GDP, 300 μg/mL saponin, 0.1% BSA). The compound of each example was added to a cell membrane fraction containing 1.8 to 3 μg/assay of membrane protein on a plate, followed by incubation at room temperature for 30 minutes. Thereafter, with glutamic acid (in a final concentration of 10 μM) added thereto, incubation was performed at room temperature for 15 minutes, and thereafter, [³⁵S]GTPγS (in a final concentration of 0.8 kBq) and 588 μg WGA-SPA beads were added thereto, followed by incubation at room temperature for 1 hour. After the incubation, the plate was subjected to the centrifugal separation at 2,500 rpm and room temperature, and then, membrane cell binding radioactivity was measured with a top count.

A [³⁵S]GTPγS binding amount obtained by performing the above-described reaction in the absence of glutamic acid was defined as nonspecific binding, and a difference from a [³⁵S]GTPγS binding amount obtained in the presence of glutamic acid was defined as specific binding. On the basis of ratios of inhibiting the specific binding at various concentrations of the compounds of the respective examples, inhibition curves were obtained. Concentrations of the compounds of the respective examples at which the specific [³⁵S]GTPγS binding amount was suppressed by 50% (IC50 values) were calculated on the basis of the inhibition curves and shown in Table 3.

TABLE 3

| | GTPγS |
|---|---|
| | binding |
| Example | assay |
| No. | IC₅₀ (nM) |
| 1 | 3.7 |
| 2 | 3.1 |
| 3 | 5.0 |
| 4 | 3.3 |
| 5 | 2.7 |
| 6 | 3.2 |
| 7 | 3.6 |
| 8 | 3.1 |
| 9 | 2.7 |
| 10 | 1.8 |
| 11 | 7.1 |
| 12 | 1.4 |
| 13 | 4.0 |
| 14 | 9.8 |
| 15 | 4.1 |
| 16 | 4.2 |
| 17 | 2.3 |
| 18 | 3.6 |
| 19 | 6.3 |
| 20 | 21.8 |
| 21 | 11.2 |
| 22 | 3.1 |
| 23 | 1.6 |
| 24 | 2.7 |
| 25 | 3.1 |
| 26 | 3.7 |
| 27 | 2.3 |
| 28 | 7.4 |
| 29 | 26.8 |
| 30 | 7.4 |
| 31 | 3.0 |
| 32 | 3.9 |
| 33 | 3.0 |
| 34 | 7.0 |
| 35 | 1.8 |
| 36 | 1.3 |
| 37 | 2.3 |
| 38 | 2.2 |
| 39 | 0.9 |
| 40 | 2.6 |
| 41 | 2.1 |
| 42 | 9.8 |
| 43 | 3.1 |
| 44 | 2.3 |
| 45 | 2.2 |
| 46 | 6.0 |
| 47 | 6.3 |
| 48 | 3.6 |
| 49 | 11.2 |
| 50 | 9.5 |
| 51 | 11.9 |
| 52 | 4.1 |
| 53 | 4.7 |
| 54 | 4.9 |
| 55 | 2.9 |
| 56 | 10.7 |
| 57 | 4.1 |
| 58 | 15.3 |
| 59 | 3.9 |
| 60 | 4.6 |
| 61 | 3.7 |
| 62 | 4.3 |
| 63 | 3.9 |
| 64 | 4.5 |
| 65 | 6.7 |
| 66 | 10.9 |

### Test Example 2

### Novel Object Recognition (NOR) Test in Rats

Six-week old male Long-Evans rats were used for this test. For 2 days before starting the test, the rats were acclimated to experimental operations such as administration and a test device (that is, a black or gray plastic cage with a width of 40 cm, a depth of 30 cm and a height of 45 cm). Each test compound was dissolved in a 0.1 N hydrochloric acid to be orally administered. Thirty minutes after the administration, scopolamine hydrobromide was intraperitoneally administered at a 0.3 mg/kg dose, so as to induce cognitive impairment. After another 30 minutes, each rat was acclimated in the test device for 3 minutes, and thereafter, two blocks in the same shape were put in the test device as acquisition trial, and exploring time for each block was measured for 5 minutes. Two hours after the acquisition trial, the rat was acclimated in the test device for 3 minutes, and thereafter, the same block as those used in the acquisition trial and a new block in a different shape were put in the cage for retention trial. The exploring time for each block was measured for 3 minutes, and a ratio of the exploring time for the newly used block to the sum of the exploring times for the respective blocks was calculated as a discrimination index. The thus obtained discrimination indexes were compared among a group of rats to which a medium alone was administered (medium group), a group of rats to which scopolamine alone was administered (scopolamine alone group) and a group of rats to which both the test compound and scopolamine were administered, so as to evaluate the action of the test compound on the novel object recognition function (cognitive function) of the rats.

Each discrimination index was shown as an average and a standard error. The statistical significance between the medium group and the scopolamine alone group was analyzed by the independent t-test. The statistical significance between the scopolamine alone group and each sample group was analyzed by one-way analysis of variance and then by Dunnett's multiple comparison test. The significance level was set to 5% on both sides. If the discrimination index was significantly lower in the scopolamine alone group than in the medium group, it was determined that the cognitive impairment was sufficiently induced, and hence, the test compound was evaluated in the corresponding group. The analysis was carried out by using Prism 5 for Windows for Japanese, ver. 5.03. A minimum effective dose at which a statistically significant difference was found between a group suffering from the cognitive impairment induced by scopolamine and a group treated with each compound is shown in Table 4.

| Example | Minimum effective dose |
|---|---|
| No. | (mg/kg, p.o.) |
| 1 | 1 |
| 2 | 3 |
| 3 | 1 |
| 8 | 3 |

What is claimed is:

1. A compound represented by formula (I): 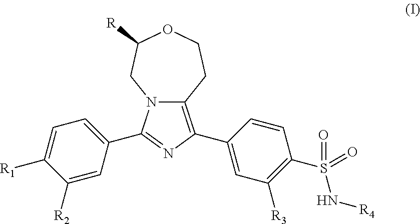
wherein
R is a methyl group or a fluoromethyl group,
R₁ is a fluorine atom, a methoxy group, an ethoxy group, a fluoromethyloxy group, a difluoromethyloxy group, or an oxetan-3-yloxy group,
R₂ is a hydrogen atom or a fluorine atom,
R₃ is a hydrogen atom,
R₄ is a methyl group, an ethyl group, an isopropyl group, a tert-butyl group, a cyclopropyl group, a cyclobutyl group, or a 1-methylcyclobutyl group,
or a pharmaceutically acceptable salt thereof.

2. A compound selected from the following compounds or a pharmaceutically acceptable salt thereof:
(R)—N-isopropyl-4-(3-(4-methoxyphenyl)-6-methyl-5,6,8,9-tetrahydroimidazo[1,5-d][1,4]oxazepin-1-yl)benzenesulfonamide,
(R)—N-cyclobutyl-4-(3-(4-methoxyphenyl)-6-methyl-5,6,8,9-tetrahydroimidazo[1,5-d][1,4]oxazepin-1-yl)benzenesulfonamide,
(R)-4-(3-(4-fluorophenyl)-6-methyl-5,6,8,9-tetrahydroimidazo[1,5-d][1,4]oxazepin-1-yl)-N-isopropylbenzenesulfonamide,
(S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide, and
(R)—N-isopropyl-4-(3-(3-fluoro-4-methoxyphenyl)-6-methyl-5,6,8,9-tetrahydroimidazo[1,5-d][1,4]oxazepin-1-yl)benzenesulfonamide.

3. (R)—N-cyclobutyl-4-(3-(4-methoxyphenyl)-6-methyl-5,6,8,9-tetrahydroimidazo[1,5-d][1,4]oxazepin-1-yl)benzenesulfonamide represented by the following formula: 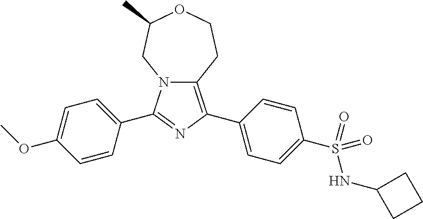 [SMILES: COc1ccc(-c2nc(-c3ccc(S(=O)(=O)NC4CCC4)cc3)c3n2C[C@@H](C)OCC3)cc1]
or a pharmaceutically acceptable salt thereof.

4. (R)-4-(3-(4-fluorophenyl)-6-methyl-5,6,8,9-tetrahydroimidazo[1,5-d][1,4]oxazepin-1-yl)-N-isopropylbenzenesulfonamide represented by the following formula: 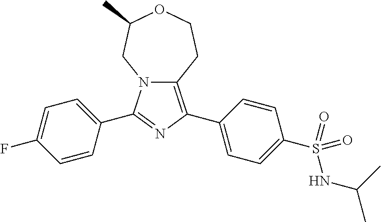 [SMILES: CC(C)NS(=O)(=O)c1ccc(-c2nc(-c3ccc(F)cc3)n3c2CCO[C@H](C)C3)cc1]
or a pharmaceutically acceptable salt thereof.

5. (S)—N-(tert-butyl)-4-(6-(fluoromethyl)-3-(4-methoxyphenyl)-5,6,8,9-tetrahydroimidazo[1,5-d][1,4]oxazepin-1-yl)benzenesulfonamide represented by the following formula: 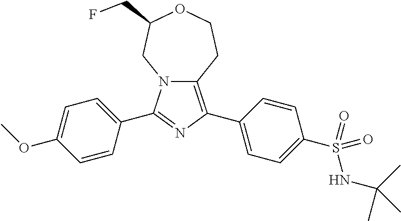 [SMILES: COc1ccc(-c2nc(-c3ccc(S(=O)(=O)NC(C)(C)C)cc3)c3n2C[C@@H](CF)OCC3)cc1]
or a pharmaceutically acceptable salt thereof.

6. A pharmaceutical composition comprising the compound or a pharmaceutically acceptable salt thereof according to claim 1, and at least one pharmaceutically acceptable excipient.

7. The pharmaceutical composition according to claim 6, for treatment of a disease or a symptom against which a group II metabotropic glutamate receptor antagonistic action is effective.

8. The pharmaceutical composition according to claim 7, wherein the disease or the symptom is Alzheimer's disease.

9. A method of treating a disease or a symptom against which a group II metabotropic glutamate receptor antagonistic action is effective, comprising administering the compound or a pharmaceutically acceptable salt thereof according to claim 1 to a subject in need thereof.

10. The method according to claim 9, wherein the disease or the symptom is Alzheimer's disease.
